(19) United States
(12) Patent Application Publication
(10) Pub. No.: US 2024/0200147 A1
(43) Pub. Date: Jun. 20, 2024

(54) Non-Small Cell Lung Cancer Diagnosis and Treatment

(71) Applicant: West Virginia University Board of Governors on Behalf of West Virginia University, Morgantown, WV (US)

(72) Inventor: Nancy Lan Guo, Morgantown, WV (US)

(21) Appl. No.: 18/517,623

(22) Filed: Nov. 22, 2023

Related U.S. Application Data
(60) Provisional application No. 63/384,696, filed on Nov. 22, 2022.

(51) Int. Cl. C12Q 1/6886; A61K 31/166; A61K 31/277; A61K 31/4706; A61K 31/517; A61P 35/00; G01N 33/574

(52) U.S. Cl. CPC C12Q 1/6886; A61K 31/166; A61K 31/277; A61K 31/4706; A61K 31/517; A61P 35/00; G01N 33/57423; C12Q 2600/106; C12Q 2600/118; C12Q 2600/136; C12Q 2600/158

(57) ABSTRACT

This invention provides a method of treating a patient having non-small cell lung cancer comprising administering to said patient a therapeutically effective amount of a compound selected from the group consisting of PD-198306 or pharmaceutically acceptable salts thereof, U-0126 or pharmaceutically acceptable salts thereof, ZM-306416 or pharmaceutically acceptable salts thereof, and PQ-401 or pharmaceutically acceptable salts thereof, for treating non-small cell lung cancer in said patient. This invention provides a method for producing an immune response in a patient having non-small cell lung cancer comprising administering to said patient a therapeutically effective amount of a compound selected from the group consisting of PD-198306, U-0126, ZM-306416, and PQ-401, for producing an immune response in said patient.

### CROSS-REFERENCE TO RELATED APPLICATION

This utility non-provisional patent application claims the benefit of priority to U.S. Provisional Patent Application Ser. No. 63/384,696, filed Nov. 22, 2022. The entire contents of U.S. Provisional Patent Application Ser. No. 63/384,696 are incorporated by reference into this utility non-provisional patent application as if fully rewritten herein.

### STATEMENT REGARDING FEDERALLY SPONSORED RESEARCH OR DEVELOPMENT

This invention was made with government support under Grant Nos. R01 LM009500, R56 LM009500, P20 RR016440 and GM130174 awarded by the National Institute of Health. The government has certain rights in the invention.

### BACKGROUND OF THE INVENTION

### 1. Field of the Invention

This invention provides a method of treating a patient having non-small cell lung cancer comprising administering to said patient an effective amount of a compound selected from the group consisting of PD-198306, U-0126, ZM-306416, and PQ-401, for treating non-small cell lung cancer in said patient or for producing an immune response and response to treatment selected from a group of 21 National Comprehensive Cancer Network (NCCN)-recommended drugs for treating non-small cell lung cancer in said patient. This invention provides a method of producing an immune response and response to treatment selected from a group of 21 NCCN-recommended drugs for treating non-small cell lung cancer in a patient having non-small cell lung cancer comprising administering to said patient a therapeutically effective amount of a compound selected from the group consisting of PD-198306, U-0126, ZM-306416, and PQ-401, for producing an immune response and response to treatment selected from a group of 21 NCCN-recommended drugs for treating non-small cell lung cancer in said patient. In addition, this invention provides a method of diagnosis and screening of lung cancer using a 7-gene assay.

### 2. Background Art

There are currently no effective biomarkers for prognosis and optimal treatment selection to improve non-small cell lung cancer (NSCLC) survival outcomes.

A seven-gene prognostic panel is set forth in U.S. patent application Ser. No. 17/251,359, filed Dec. 11, 2020, and published as US Patent Application Publication Serial No. US 2021-0254173 A1 on Aug. 19, 2021, entitled Predictive 7-Gene Assay and Prognostic Protein Biomarker For Non-Small Cell Lung Cancer, is incorporated by reference herein, for predicting the risk of tumor recurrence and metastasis in non-small cell lung cancer as understood by those persons of ordinary skill in the art.

A seven-gene prognostic and predictive assay for Non-Small cell Lung Cancer is set forth in U.S. patent application Ser. No. 17/906,315, filed Sep. 14, 2022, and published as US Patent Application Publication Serial No. US 2023/0106465 A1 on Apr. 6, 2023, entitled 7-Gene Prognostic and Predictive Assay For Non-Small Cell Lung cancer in Formalin Fixed and Paraffin Embedded Samples, is incorporated by reference herein, for background methodology and ABCC4, CCL19, SLC39A8, CD27, FUT7, ZNF71 and DAG1 and including SEQ ID for gene and protein, as understood by those persons of ordinary skill in the art.

Artificial intelligence (AI) methodology for drug discovery is set forth in Patent Cooperation Treaty (PCT) Patent Application Serial No. PCT/US2022/075136 having an International Filing Date of Aug. 18, 2022 and published as WO 2023/023592 on Feb. 23, 2023, entitled Repurposing Drugs For Non-Small Cell Lung Cancer, is incorporated by reference herein, for background data and methodology as understood by those persons of ordinary skill in the art.

### SUMMARY OF THE INVENTION

A method of treating a patient having non-small cell lung cancer comprises administering to said patient a therapeutically effective amount of a compound selected from the group consisting of PD-198306, U-0126, ZM-306416, and PQ-401, for treating non-small cell lung cancer in said patient. In certain embodiments of this method, the compound is PD-198306 or pharmaceutically acceptable salts thereof. In certain embodiments of this method, the compound is U-0126 or pharmaceutically acceptable salts thereof. In certain embodiments of this method, the compound is ZM-306416 or pharmaceutically acceptable salts thereof. In certain embodiments of this method, the compound is PQ-401 or pharmaceutically acceptable salts thereof.

Another embodiment of this invention provides a method of treating a patient having non-small cell lung cancer comprising administering to said patient a therapeutically effective amount of a composition comprising a compound selected from the group consisting of PD-198306, U-0126, ZM-306416, and PQ-401, and a therapeutically acceptable pharmaceutical carrier, for treating non-small cell lung cancer in said patient.

In another embodiment of this invention, a method of producing an immune response and response to treatment selected from a group of 21 NCCN-recommended drugs for treating non-small cell lung cancer in a patient having non-small cell lung cancer is provided comprising administering to said patient a therapeutically effective amount of a compound selected from the group consisting of PD-198306, U-0126, ZM-306416, and PQ-401, for producing an immune response and response to treatment selected from a group of 21 NCCN-recommended drugs for treating non-small cell lung cancer in said patient. In certain embodiments of this method, the compound is PD-198306. In certain embodiments of this method, the compound is U-0126. In certain embodiments of this method, the compound is U-0126. In certain embodiments of this method, the compound is ZM-306416. In certain embodiments of this method, the compound is PQ-401.

Another embodiment of this invention provides a method for producing an immune response and response to treatment selected from a group of 21 NCCN-recommended drugs for treating non-small cell lung cancer in a patient having non-small cell lung cancer comprising administering to said patient a therapeutically effective amount of a composition comprising a compound selected from the group consisting of PD-198306, U-0126, ZM-306416, and PQ-401, and a therapeutically acceptable pharmaceutical carrier, for producing an immune response in said patient.

A method of treating a patient having non-small cell lung cancer is provided comprising administering to said patient a therapeutically effective amount of a compound that is an MEK1/2 inhibitor selected from the group consisting of PD-198306 and U-0126.

A method of treating a patient having non-small cell lung cancer is provided comprising administering to said patient a therapeutically effective amount of a composition comprising a compound that is a MEK1/2 inhibitor selected from the group consisting of PD-198306 and U-0126, and a therapeutically acceptable pharmaceutical carrier.

A method of treating a patient having non-small cell lung cancer is provided comprising administering to said patient a therapeutically effective amount of a compound that is a VEGFR inhibitor that is ZM-306416.

A method of treating a patient having non-small cell lung cancer is provided comprising administering to said patient a therapeutically effective amount of a composition comprising a compound that is a VEGFR inhibitor that is ZM-306416, and a therapeutically acceptable pharmaceutical carrier for treating a patient having non-small cell lung cancer.

A method of treating a patient having non-small cell lung cancer is provided comprising administering to said patient a therapeutically effective amount of a compound that is an IGF-1R inhibitor that is PQ-401.

A method of treating a patient having non-small cell lung cancer is provided comprising administering to said patient a therapeutically effective amount of a composition comprising a compound that is an IGF-1R inhibitor that is PQ-401 and a therapeutically acceptable pharmaceutical carrier for treating a patient having non-small cell lung cancer.

A method of diagnosis and screening of non-small cell lung cancer using protein expression levels of ABCC4, DAG1, and SLC39A8 in patient samples.

A method of diagnosis and screening of non-small cell lung cancer using protein expression levels of ABCC4 and DAG1 in patient samples.

A method of diagnosis and screening of non-small cell lung cancer using protein expression levels of ABCC4 and SLC39A8 in patient samples.

A method of diagnosis and screening of non-small cell lung cancer using protein expression levels of DAG1 and SLC39A8 in patient samples.

A method of diagnosis and screening of non-small cell lung cancer using protein expression levels of ABCC4 in patient samples.

A method of diagnosis and screening of non-small cell lung cancer using protein expression levels of DAG1 in patient samples.

A method of diagnosis and screening of non-small cell lung cancer using protein expression levels of SLC39A8 in patient samples.

A method of diagnosis and screening of non-small cell lung cancer using mRNA expression levels of ABCC4, CCL19, CD27, DAG1, FUT7, and ZNF71 in patient samples.

A method of diagnosis and screening of non-small cell lung cancer using mRNA expression levels of the following gene combinations in patient samples.

Combinations of genes whose mRNA expression can be
used for diagnosis and screening of lung cancer.

FUT7
ABCC4 + FUT7
ZNF71 + CCL19
ZNF71 + FUT7
CD27 + CCL19
CD27 + FUT7
CCL19 + FUT7
DAG1 + FUT7
ABCC4 + ZNF71 + CD27
ABCC4 + ZNF71 + DAG1
ABCC4 + ZNF71 + CCL19
ABCC4 + ZNF71 + FUT7
ABCC4 + CD27 + DAG1
ABCC4 + CD27 + CCL19
ABCC4 + CD27 + FUT7
ABCC4 + DAG1 + CCL19
ABCC4 + DAG1 + FUT7
ABCC4 + CCL19 + FUT7
ZNF71 + CD27 + DAG1
ZNF71 + CD27 + CCL19
ZNF71 + CD27 + FUT7
ZNF71 + DAG1 + CCL19
ZNF71 + DAG1 + FUT7
ZNF71 + CCL19 + FUT7
CD27 + DAG1 + CCL19
CD27 + DAG1 + FUT7
CD27 + CCL19 + FUT7
DAG1 + CCL19 + FUT7
ABCC4 + ZNF71 + CD27 + DAG1
ABCC4 + ZNF71 + CD27 + CCL19
ABCC4 + ZNF71 + CD27 + FUT7
ABCC4 + ZNF71 + DAG1 + CCL19
ABCC4 + ZNF71 + DAG1 + FUT7
ABCC4 + ZNF71 + CCL19 + FUT7
ABCC4 + CD27 + DAG1 + CCL19
ABCC4 + CD27 + DAG1 + FUT7
ABCC4 + CD27 + CCL19 + FUT7
ABCC4 + DAG1 + CCL19 + FUT7
ZNF71 + CD27 + DAG1 + CCL19
ZNF71 + CD27 + DAG1 + FUT7
ZNF71 + CD27 + CCL19 + FUT7
ZNF71 + DAG1 + CCL19 + FUT7
CD27 + DAG1 + CCL19 + FUT7
ABCC4 + ZNF71 + CD27 + DAG1 + CCL19
ABCC4 + ZNF71 + CD27 + DAG1 + FUT7
ABCC4 + ZNF71 + CD27 + CCL19 + FUT7
ABCC4 + ZNF71 + DAG1 + CCL19 + FUT7
ABCC4 + CD27 + DAG1 + CCL19 + FUT7
ZNF71 + CD27 + DAG1 + CCL19 + FUT7

A method of using expression of CDT1 and/or INCENP, or a combination thereof, to predict sensitivity to afatinib.

A method of using expression of IL4I1, LRP1, FAM156B, DCUN1D4, ESPN, PTPRJ, and/or NMNAT2, or a combination thereof, to predict resistance to afatinib.

A method of using expression of CSNK2A3, DYNC1H1, and/or PSME1, or a combination thereof, to predict sensitivity to alectinib.

A method of using expression of UCP3, CDC45, THSD7A, ANKRD52, EIF2A, FAM156B, KRT8, and/or CFAP44, or a combination thereof, to predict resistance to alectinib.

A method of using expression of MYOF, ABHD10, and/or TMTC4, or a combination thereof, to predict resistance to brigatinib.

A method of using expression of CSNK2A3, or a combination thereof, to predict sensitivity to cabozantinib.

A method of using expression of RNASEL, TMTC4, SMC2, ASB7, CFAP44, IL17RA, ZXDA, and/or THSD7A, or a combination thereof, to predict resistance to cabozantinib.

A method of using expression of ASB7 and/or RAB1A, or a combination thereof, to predict resistance to carboplatin.

A method of using expression of ZNF507, CSNK2A3, and/or WDR17, or a combination thereof, to predict sensitivity to cisplatin.

A method of using expression of ARHGAP12, CDC45, ESPN, EIF2A, IL17RA, MYOF, TMTC4, NMNAT2, PTPRJ, and/or KRT8, or a combination thereof, to predict resistance to cisplatin.

A method of using expression of RFC4, CSNK2A3, MCM2, MDN1, CDT1, and/or INCENP, or a combination thereof, to predict sensitivity to crizotinib.

A method of using expression of LRP1 and/or IL17RA, or a combination thereof, to predict resistance to crizotinib.

A method of using expression of FBLN7, MAP2, CTNNB1, RNF128, CSNK2A3, WDR17, and/or ZNF507, or a combination thereof, to predict sensitivity to dabrafenib.

A method of using expression of FAM156B, SLC39A8, SMC2, KRT8, and/or NMNAT2, or a combination thereof, to predict resistance to dabrafenib.

A method of using expression of CDT1, or a combination thereof, to predict sensitivity to dacomitinib.

A method of using expression of MAP3K7, DCUN1D4, LRP1, THOC5, FAM156B, ANKRD52, and/or THSD7A, or a combination thereof, to predict resistance to dacomitinib.

A method of using expression of RFC4, ABCC5, FBLN7, and/or MAP2, or a combination thereof, to predict sensitivity to docetaxel.

A method of using expression of SLC39A8, IL17RA, KRT8, RAB1A, THSD7A, DCUN1D4, MYOF, and/or NMNAT2, or a combination thereof, to predict resistance to docetaxel.

A method of using expression of CSNK2A3, PSMB6, and/or TGFB2, or a combination thereof, to predict sensitivity to erlotinib.

A method of using expression of FAM156B, ASB7, TMTC4, and/or NMNAT2, or a combination thereof, to predict resistance to erlotinib.

A method of using expression of PSMB3, CDT1, DYNC1H1, and/or ZNF507, or a combination thereof, to predict sensitivity to etoposide.

A method of using expression of ESPN, NMNAT2, ZNF324B, and/or IL17RA, or a combination thereof, to predict resistance to etoposide.

A method of using expression of BRMS1L, CCNA2, CDT1, and/or INCENP, or a combination thereof, to predict sensitivity to gefitinib.

A method of using expression of IL4I1, ABHD10, FAM156B, MAP3K7, DCUN1D4, NMNAT2, PTPRJ, and/or KRT8, or a combination thereof, to predict resistance to gefitinib.

A method of using expression of MDGA1, MDN1, ZNF507, and/or WDR17, or a combination thereof, to predict sensitivity to gemcitabine.

A method of using expression of KRT8, ANKRD52, ASB7, RAB1A, FAM156B, IL17RA, DCUN1D4, IL4I1, LRP1, and/or NMNAT2, or a combination thereof, to predict resistance to gemcitabine.

A method of using expression of MDN1 and/or PSMB6, or a combination thereof, to predict sensitivity to lorlatinib.

A method of using expression of ANKRD52, ESPN, and/or RAB1A, or a combination thereof, to predict resistance to lorlatinib.

A method of using expression of ABCC5, MDGA1, TCF20, MAP2, and/or PSMB6, or a combination thereof, to predict sensitivity to osimertinib.

A method of using expression of ASB7, DCUN1D4, and/or THSD7A, or a combination thereof, to predict resistance to osimertinib.

A method of using expression of INCENP, CDT1, MCM2, and/or TCF20, or a combination thereof, to predict sensitivity to paclitaxel.

A method of using expression of RAB1A, THOC5, ASB7, IL17RA, NMNAT2, KRT8, LRP1, SLC39A8, THSD7A, and/or UCP3, or a combination thereof, to predict resistance to paclitaxel.

A method of using expression of MCM2 and/or CCNA2, or a combination thereof, to predict sensitivity to pemetrexed.

A method of using expression of EIF2A, IL17RA, MYOF, THSD7A, ZXDA, ARHGAP12, and/or LRP1, or a combination thereof, to predict resistance to pemetrexed.

A method of using expression of CTNNB1, or a combination thereof, to predict sensitivity to trametinib.

A method of using expression of RNASEL, IL17RA, ANKRD52, FAM156B, AP2S1, ARHGAP12, SRRM2, UCP3, ZXDA, TMTC4, DCUN1D4, NCOR1, and/or RAB1A, or a combination thereof, to predict resistance to trametinib.

A method of using expression of RNASE L and/or IL17RA , or a combination thereof, to predict resistance to vemurafenib.

A method of using expression of ABCC5, DYNC1H1, IRX1, and/or TCF20, or a combination thereof, to predict sensitivity to vinorelbine.

A method of using expression of RAB1A, SLC39A8, KRT8, DCUN1D4, PTPRJ, IL17RA, MYOF, and/or NMNAT2, or a combination thereof, to predict resistance to vinorelbine.

### BRIEF DESCRIPTION OF THE DRAWINGS

FIG. 1A shows the prognosis of the seven-gene panel using RNA sequencing and proteomic profiles in NSCLC tumors. Kaplan-Meier analysis and log₂ ratio of xCell scores of the high-risk group vs. the low-risk group using three-gene (ABCC4, SLC39A8, and DAG1) cox model in LUAD proteomics data.

FIG. 1B shows the prognosis of the seven-gene panel using RNA sequencing and proteomic profiles in NSCLC tumors. Kaplan-Meier analysis and log₂ ratio of xCell scores of the high-risk group vs. the low-risk group using RNA sequencing data of ABCC4 in TCGA-NSCLC.

FIG. 1C shows the prognosis of the seven-gene panel using RNA sequencing and proteomic profiles in NSCLC tumors. Kaplan-Meier analysis and log₂ ratio of xCell scores of the high-risk group vs. the low-risk group using proteomics data of ABCC4 in LUAD [33].

FIG. 1D shows the prognosis of the seven-gene panel using RNA sequencing and proteomic profiles in NSCLC tumors. Kaplan-Meier analysis and log₂ ratio of xCell scores of the high-risk group vs. the low-risk group using proteomics data of CCL19 in LUAD [33].

FIG. 1E shows the prognosis of the seven-gene panel using RNA sequencing and proteomic profiles in NSCLC tumors. Kaplan-Meier analysis and log₂ ratio of xCell scores of the high-risk group vs. the low-risk group using proteomics data of DAG1 in LUAD [33].

FIG. 1F shows the prognosis of the seven-gene panel using RNA sequencing and proteomic profiles in NSCLC tumors. Kaplan-Meier analysis and log₂ ratio of xCell scores of the high-risk group vs. the low-risk group using RNA sequencing data of DAG1 in LUAD [33].

FIG. 1G shows the prognosis of the seven-gene panel using RNA sequencing and proteomic profiles in NSCLC tumors. Kaplan-Meier analysis and log₂ ratio of xCell scores of the high-risk group vs. the low-risk group using RNA sequencing data of CD27 in LUAD [33].

FIG. 1H shows the prognosis of the seven-gene panel using RNA sequencing and proteomic profiles in NSCLC tumors. Kaplan-Meier analysis and log₂ ratio of xCell scores of the high-risk group vs. the low-risk group using proteomics data of SLC39A8 in LUAD [33].

FIG. 2A shows the classification of non-cancerous adjacent tissues (NATs) and lung adenocarcinoma (LUAD) tumors with the seven-gene panel Log₂ ratio of xCell scores of the LUAD tumor vs. NATs RNA sequencing data. Two sample t-tests were performed to test the difference between the two groups (*: p<0.05; **: p<0.01; ***: p<0.001.

FIG. 2B shows the classification of non-cancerous adjacent tissues (NATs) and lung adenocarcinoma (LUAD) tumors with the seven-gene panel boxplots of available RNA sequencing gene expression.

FIG. 2C shows the classification of non-cancerous adjacent tissues (NATs) and lung adenocarcinoma (LUAD) tumors with the seven-gene panel principal component analysis (PCA) of RNA sequencing gene expression.

FIG. 2D shows the classification of non-cancerous adjacent tissues (NATs) and lung adenocarcinoma (LUAD) tumors with the seven-gene panel protein expression in tumors and NATs of the 7-gene panel in LUAD patients [33].

FIG. 2E shows the classification of non-cancerous adjacent tissues (NATs) and lung adenocarcinoma (LUAD) tumors with the seven-gene panel protein expression in separating NATs and LUAD tumors in patients.

FIG. 3A shows the association of ZNF71 and xCell scores of selected immune cells. Prognostic implications of ZNF71 and xCell scores of dendritic cells in TCGA NSCLC patient tumors. Kaplan-Meier analysis of TCGA-NSCLC patients stratified by the median value of ZNF71 mRNA expression.

FIG. 3B shows the association of ZNF71 and xCell scores of selected immune cells. Prognostic implications of ZNF71 and xCell scores of dendritic cells in TCGA NSCLC patient tumors. Kaplan-Meier analysis of TCGA-NSCLC patients stratified by the median value of ZNF71 mRNA expression and the median value of dendritic cells (DC) xCell scores.

FIG. 3C shows the association of ZNF71 and xCell scores of selected immune cells. Prognostic implications of ZNF71 and xCell scores of dendritic cells in TCGA NSCLC patient tumors. χ² test results of ZNF71 expression vs. xCell scores of NK cells and NKT in TCGA-NSCLC patient tumors.

FIG. 4 shows that ZNF71 suppresses components of the innate and intrinsic immune response. Western blotting of control RFP, ZNF71 KRAB, and KRABless isoforms overexpression, as well as of several markers of intracellular innate and intrinsic immune systems in A549 cells. GAPDH is shown as the loading control.

FIG. 5A shows direct gene co-expression networks of ZNF71 and intracellular innate immune response (IIIR) genes in lung adenocarcinoma (LUAD) patient samples using RNA sequencing data. Direct gene associations of ZNF71 and IIIR genes when ZNF71 is upregulated in LUAD tumors.

FIG. 5B shows direct gene co-expression networks of ZNF71 and intracellular innate immune response (IIIR) genes in lung adenocarcinoma (LUAD) patient samples using RNA sequencing data. Direct gene associations of ZNF71 and IIIR genes when ZNF71 is upregulated in NATs.

FIG. 5C shows direct gene co-expression networks of ZNF71 and intracellular innate immune response (IIIR) genes in lung adenocarcinoma (LUAD) patient samples using RNA sequencing data. Direct gene associations of ZNF71 and IIIR genes when ZNF71 is downregulated in NATs.

FIG. 6A shows indirect gene co-expression networks of ZNF71 and intracellular innate immune response (IIIR) genes in lung adenocarcinoma (LUAD) patient samples using RNA sequencing data. Gene associations of ZNF71 with IIIR genes through intermediate genes in LUAD tumors when ZNF71 is upregulated.

FIG. 6B shows indirect gene co-expression networks of ZNF71 and intracellular innate immune response (IIIR) genes in lung adenocarcinoma (LUAD) patient samples using RNA sequencing data. Gene associations of ZNF71 with IIIR genes through intermediate genes in NATs when ZNF71 is downregulated.

FIG. 6C shows indirect gene co-expression networks of ZNF71 and intracellular innate immune response (IIIR) genes in lung adenocarcinoma (LUAD) patient samples using RNA sequencing data. Gene associations of ZNF71 with IIIR genes through intermediate genes in NATs when ZNF71 is upregulated.

FIG. 7A shows multi-omics gene association networks of ZNF71 and intracellular innate immune response (IIIR) genes in lung adenocarcinoma (LUAD) patient samples using RNA sequencing and proteomics data. Gene associations between ZNF71 and IIIR genes through intermediate genes in LUAD tumors when ZNF71 is upregulated.

FIG. 7B shows multi-omics gene association networks of ZNF71 and intracellular innate immune response (IIIR) genes in lung adenocarcinoma (LUAD) patient samples using RNA sequencing and proteomics data. Gene associations between ZNF71 and IIIR genes through intermediate genes in NATs when ZNF71 is upregulated.

FIG. 7C shows multi-omics gene association networks of ZNF71 and intracellular innate immune response (IIIR) genes in lung adenocarcinoma (LUAD) patient samples using RNA sequencing and proteomics data. Gene associations between ZNF71 and IIIR genes through intermediate genes in LUAD tumors when ZNF71 is downregulated.

FIG. 7D shows multi-omics gene association networks of ZNF71 and intracellular innate immune response (IIIR) genes in lung adenocarcinoma (LUAD) patient samples using RNA sequencing and proteomics data. Gene associations between ZNF71 and IIIR genes through intermediate genes in NATs when ZNF71 is downregulated.

FIG. 8A shows discovering repositioning drugs based on the selected genes. Selection of significant functional pathways and repositioning drugs based on the selected genes.

FIG. 8B shows discovering repositioning drugs based on the selected genes. The Pearson correlations of PDL1 (CD274) protein expression with AS-703026 EC₅₀ excluding outliers.

FIG. 8C shows discovering repositioning drugs based on the selected genes. CTLA4 mRNA expression with PD-198306 IC₅₀ excluding outliers.

FIG. 8D shows discovering repositioning drugs based on the selected genes. PD-198306 ln(IC₅₀) excluding outliers.

FIG. 8E shows discovering repositioning drugs based on the selected genes. ZM-306416 ln(EC₅₀) excluding outliers.

FIG. 8F shows discovering repositioning drugs based on the selected genes. Selumetinib ln(IC₅₀).

FIG. 8G shows discovering repositioning drugs based on the selected genes. CD27 mRNA expression with PQ-401 ln(EC₅₀) excluding outliers.

FIG. 8H shows discovering repositioning drugs based on the selected genes. U-0126 ln(IC₅₀).

FIG. 8I shows discovering repositioning drugs based on the selected genes. PD1(PDCD1) mRNA expression with PQ-401 ln(EC₅₀) excluding outliers.

FIG. 8J shows discovering repositioning drugs based on the selected genes. Selected compound that had a low average IC₅₀ and EC₅₀ in the CCLE NSCLC cell lines (n=64 [IC₅₀]; n=88 [EC_{50]}).

### DETAILED DESCRIPTION OF THE INVENTION

This invention provides a method of treating a patient having non-small cell lung cancer comprising administering to said patient a therapeutically effective amount of a compound selected from the group consisting of PD-198306, U-0126, ZM-306416, and PQ-401, for treating non-small cell lung cancer in said patient. In certain embodiments of this method, the compound is PD-198306 or pharmaceutically acceptable salts thereof. In certain embodiments of this method, the compound is U-0126 or pharmaceutically acceptable salts thereof. In certain embodiments of this method, the compound is ZM-306416 or pharmaceutically acceptable salts thereof. In certain embodiments of this method, the compound is PQ-401 or pharmaceutically acceptable salts thereof.

Another embodiment of this invention provides a method of treating a patient having non-small cell lung cancer comprising administering to said patient a therapeutically effective amount of a composition comprising a compound selected from the group consisting of PD-198306 or pharmaceutically acceptable salts thereof, U-0126 or pharmaceutically acceptable salts thereof, ZM-306416 or pharmaceutically acceptable salts thereof, and PQ-401 or pharmaceutically acceptable salts thereof, and a therapeutically acceptable pharmaceutical carrier, for treating non-small cell lung cancer in said patient.

In another embodiment of this invention, a method of producing an immune response in a patient having non-small cell lung cancer is provided comprising administering to said patient a therapeutically effective amount of a compound selected from the group consisting of PD-198306, U-0126, ZM-306416, and PQ-401, for producing an immune response in said patient. In certain embodiments of this method, the compound is PD-198306. In certain embodiments of this method, the compound is U-0126. In certain embodiments of this method, the compound is U-0126. In certain embodiments of this method, the compound is ZM-306416. In certain embodiments of this method, the compound is PQ-401.

Another embodiment of this invention provides a method for producing an immune response in a patient having non-small cell lung cancer comprising administering to said patient a therapeutically effective amount of a composition comprising a compound selected from the group consisting of PD-198306, U-0126, ZM-306416, and PQ-401, and a therapeutically acceptable pharmaceutical carrier, for producing an immune response in said patient.

A method of treating a patient having non-small cell lung cancer is provided comprising administering to said patient a therapeutically effective amount of a compound that is a MEK1/2 inhibitor selected from the group consisting of PD-198306 and U-0126.

A method of treating a patient having non-small cell lung cancer is provided comprising administering to said patient a therapeutically effective amount of a composition comprising a compound that is a MEK1/2 inhibitor selected from the group consisting of PD-198306 and U-0126, and a therapeutically acceptable pharmaceutical carrier.

A method of treating a patient having non-small cell lung cancer is provided comprising administering to said patient a therapeutically effective amount of a compound that is a VEGFR inhibitor that is ZM-306416.

A method of treating a patient having non-small cell lung cancer is provided comprising administering to said patient a therapeutically effective amount of a composition comprising a compound that is a VEGFR inhibitor that is ZM-306416, and a therapeutically acceptable pharmaceutical carrier for treating a patient having non-small cell lung cancer.

A method of treating a patient having non-small cell lung cancer is provided comprising administering to said patient a therapeutically effective amount of a compound that is a IGF-1R inhibitor that is PQ-401.

A method of treating a patient having non-small cell lung cancer is provided comprising administering to said patient a therapeutically effective amount of a composition comprising a compound that is a IGF-1R inhibitor that is PQ-401 and a therapeutically acceptable pharmaceutical carrier for treating a patient having non-small cell lung cancer.

The methods of this invention include the following compounds:

| Name | PubChem Formula | Name and Address of a Source(s) |
|---|---|---|
| U-0126 | 3006531 C18H16N6S2 | Tocris Bioscience, Bio-Techne Corporation, 614 Mckinley Place NE, Minneapolis, MN 55413, USA |
| ZM-306416 | 5329006 C16H13ClFN3O2 | MyBioSource, Inc., P.O. Box 153308, San Diego, CA 92195-3308, USA |
| PQ-401 | 9549305 C18H16ClN3O2 | Tocris Bioscience, Bio-Techne Corporation, 614 Mckinley Place NE, Minneapolis, MN 55413, USA |
| PD-198306 | 9956637 C18H16F3IN2O2 | Tocris Bioscience, Bio-Techne Corporation, 614 Mckinley Place NE, Minneapolis, MN 55413, USA |

Compound U-0126 has the formula: 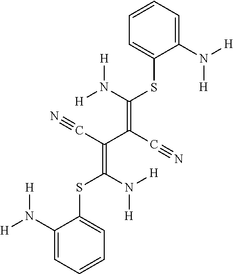 [SMILES: N#CC(=C(/N)Sc1ccccc1N)/C(C#N)=C(\N)Sc1ccccc1N]
Compound ZM-306416 has the formula: 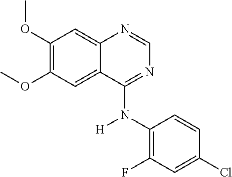 [SMILES: COc1cc2ncnc(Nc3ccc(Cl)cc3F)c2cc1OC]
Compound PQ-401 has the formula: 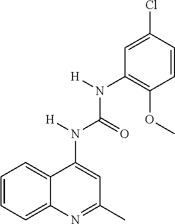 [SMILES: COc1ccc(Cl)cc1NC(=O)Nc1cc(C)nc2ccccc12]
Compound PD-198306 has the formula:

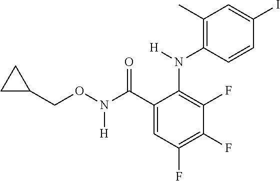 [SMILES: Cc1cc(I)ccc1Nc1c(C(=O)NOCC2CC2)cc(F)c(F)c1F]

This invention provides seven-gene panel for diagnosis and prognosis of NSCLC using RNA sequencing and proteomic profiles of patient tumors. Within the seven-gene panel, ZNF71 expression combined with dendritic cell activities defines NSCLC patient subgroups (n=966) with distinct survival outcomes (p=0.04, Kaplan-Meier analysis). ZNF71 expression is significantly associated with the activities of natural killer cells (p=0.014) and natural killer T cells (p=0.003) in NSCLC patient tumors (n=1016) using Chi-squared tests. Overexpression of ZNF71 results in decreased expression of multiple components of the intracellular intrinsic and innate immune systems, including dsRNA and dsDNA sensors. Multi-omics networks of ZNF71 and the intracellular intrinsic and innate immune systems were computed as relevant to NSCLC tumorigenesis, proliferation, and survival using patient clinical information and in-vitro CRISPR-Cas9/RNAi screening data. From these networks, pan-sensitive and pan-resistant genes to 21 NCCN-recommended drugs for treating NSCLC were selected. Based on the gene associations with patient survival and in-vitro CRISPR-Cas9, RNAi, and drug screening data, MEK1/2 inhibitors PD-198306 and U-0126, VEGFR inhibitor ZM-306416, and IGF-1R inhibitor PQ-401 were discovered as targeted therapy that may also induce immune response for treating NSCLC.

As used herein, the term “patient” means members of the animal kingdom, including but not limited to, human beings.

As used herein, the term “effective amount” or “therapeutically effective amount” refers to that amount of any of the present compounds, salts thereof, and/or compositions required to bring about a desired effect in a patient. The desired effect will vary depending upon the illness or disease state being treated. For example, the desired effect may be reducing the tumor size, destroying cancerous cells, and/or preventing metastasis, any one of which may be the desired therapeutic response. On its most basic level, a therapeutically effective amount is that amount of a substance needed to inhibit mitosis of a cancerous cell. As used herein, “tumor” refers to an abnormal growth of cells or tissues of the malignant type, unless otherwise specifically indicated and does not include a benign type tissue. The “tumor” may be comprised of at least one cell and/or tissue. The term “inhibits or inhibiting” as used herein means reducing growth/replication. As used herein, the term “cancer” refers to any type of cancer, including for example but not limited to, non-small cell lung cancer, and the like.

The methods and novel compounds and pharmaceutically acceptable salts thereof of this invention provide for treatment of tumors, or other cancer cells, in cancer patients. The types of cancer can vary widely and in certain embodiments, the methods and novel compounds and pharmaceutically acceptable salts thereof of this invention are particularly useful for example, in treating patients with non-small cell lung cancer.

As used herein, the term “therapeutically effective pharmaceutical carrier” refers to any pharmaceutically acceptable carrier known in the art, absent compatibility problems with the novel compounds of the invention. Generally, therapeutically effective pharmaceutical carriers include for example but not limited to, physiologic saline and 5% dextrose in water.

As will be understood by one skilled in the art, a therapeutically effective amount of said compound can be administered by any means known in the art, including but not limited to, injection, parenterally, intravenously, intraperitoneally, orally or, where appropriate, topically.

It is well within the skill of one practicing in the art to determine what dosage, and the frequency of this dosage, which will constitute a therapeutically effective amount for each individual patient, depending on the severity or progression of cancer or cancer cells and/or the type of cancer. It is also within the skill of one practicing in the art to select the most appropriate method of administering the compounds based upon the needs of each patient.

### 1. Introduction

Non-small cell lung cancer (NSCLC) has the second-highest cancer incidence rate and the highest cancer mortality rate for both men and women [1]. The major histological subtypes of NSCLC are lung adenocarcinoma (LUAD, 40% of NSCLC cases), squamous cell carcinoma (LUSC, 25-30%), and large cell carcinoma (LCC, 5-10%). Each subtype represents a distinct prognosis for patients and informs treatment options [2, 3]. According to the current NCCN standard of care [4], stage 1A NSCLC patients do not receive adjuvant therapy after surgery. Osimertinib is recommended for stage 1B patients with EGFR exon 19 deletion or L858R. Adjuvant therapy is recommended for patients in stage 1B with high-risk features, i.e., tumor size>4 cm, poor differentiation, vascular invasion, wedge resection, visceral pleural involvement, and unknown lymph node status, stage 2, and above. Patients in stages 3 and 4 receive additional radiotherapy [5]. Programmed Death 1 (PD-1) and its ligand PD-L1 compromise anti-tumor immunity while maintaining peripheral tolerance [6]. Anti-PD-L1 antibodies have revolutionized cancer immunotherapy, including NSCLC treatment [7]. NCCN guidelines [4] recently changed to establish adjuvant anti-PD-L1 immunotherapy (atezolizumab) after chemotherapy as the standard of care for stages 2/3A NSCLC patients with PD-L1>1%, following the FDA approval [8]. Yet, resectable NSCLC has a 5-year mortality rate of 40% in stage 1, 66% in stage 2, and 85% in stage 3A because of recurrence [9-13]. At present, there are no accurate prognostic tests that predict post-surgical recurrence/metastasis or inform the clinical benefits of adjuvant therapies, including chemotherapy and immunotherapy, in early-stage NSCLC patients—a significant unmet clinical need.

We recently discovered a seven-gene (ABCC4, CCL19, CD27, DAG1, FUT7, SLC39A8, and ZNF71) signature that accurately predicts the risk of recurrence/metastasis in retrospective analyses of 1,500 early-stage NSCLC patients for all histological subtypes, including clinical trials [14, 15]. Employing novel artificial intelligence (AI) methods and confirmed with qRT-PCR using frozen tumors (n=331) [14], our assay also predicts the clinical benefits of receiving adjuvant chemotherapy in both training and validation sets, including a clinical trial JBR.10. Results from our seven-gene panel were corroborated in The Cancer Genome Atlas (TCGA) cohort for risk stratification of early-stage NSCLC patients (n=923) [15]. Within this seven-gene panel, CD27 is a target for immune checkpoint inhibitors (ICIs) [16], and anti-CD27 mAb is being tested as an adjuvant immunotherapy in phase I/II clinical trials for multiple tumor types with promising results [17, 18]. Within the seven-gene panel, ZNF71 protein expression quantified with AQUA produced robust patient stratification in two separate NSCLC cohorts (n=191) in tissue microarrays [14]. We previously reported that the ZNF71 KRAB isoform was associated with epithelial-to-mesenchymal (EMT) transition and poor prognosis in NSCLC patients [19].

ZNF71 is a member of a large family of KRAB zinc finger transcription factors, KRAB-ZNFs, which due to the presence of the KRAB domain function as transcriptional repressors. One of the main roles ascribed to KRAB-ZNFs is the repression of retrotransposon class repetitive elements (TEs) [20] that comprise up to 36% of the human genome [21]. Retro TEs are remnants of ancient invaded viruses, which could produce dsRNA molecules and RNA/DNA hybrids. Although the vast majority of TEs in the human genome are inactivated by mutations, a small number of full-length functional elements including long interspersed nuclear elements (LINEs) and human endogenous retroviruses (HERVs) are capable of retrotransposition, mimicking viral infection. While normally silenced, they could be reactivated in cancer or response to therapy [22, 23]. The majority of cellular dsRNA is the result of the TEs transcription [24]. Non-degraded dsRNAs are recognized by specific proteins of the intracellular innate immune system also called pattern recognition receptors (PRRs) such as MDA5/IFIH1 and others, ultimately leading to a Type I interferon production [25]. Acting as specific dsRNA sensors downstream or in parallel with PRRs, the OAS-RNase L and the PKR/EIF2AK2 pathways degrade endogenous and viral dsRNA and block global cellular translation, respectively [26, 27]. In addition, a growing number of host restriction factors of the intrinsic immune system can be engaged in anti-viral response, including SAMHD1, TRIM5a, MX, and IFITM proteins [28].

Most human tumors display chromosomal instability (CIN) phenotype and aneuploidy, which are often accompanied by the generation of micronuclei and the presence of cytosolic dsDNA. Cytosolic dsDNA activates the cGAS-STING signaling pathway [29]. STING facilitates activation of TBK1 kinase leading to its autophosphorylation on S172 [30] and subsequent phosphorylation of IRF3 transcription factor, which in turn activates interferon response genes.

In this study, we sought to (1) further evaluate the diagnostic and prognostic implications of the seven-gene panel using both RNA-sequencing and proteomic profiles in diverse NSCLC patient cohorts and examine the associated immune cell activities during NSCLC tumorigenesis and progression; (2) investigate the functional involvement of ZNF71 KRAB in innate immunity; (3) identify molecular networks mediated by ZNF71 relevant to innate immunity in NSCLC tumors and normal adjacent lung tissues using a novel AI technology based on Boolean implication networks; (4) discover pan-sensitive and pan-resistant genes to a panel of 21 NCCN recommended drugs for treating NSCLC from the above identified molecular association networks; and (5) explore therapeutic compounds as new or repositioning drugs for treating NSCLC for designed mechanisms of actions based on our analysis of patient tumor profiles and in-vitro CRISPR-Cas9/RNAi and drug screening data using Connectivity Map (CMap) [31, 32].

### 2. Results

### 2.1. Further Validation of the Seven-Gene Signature in Prognosis for NSCLC

Our previous work [14] developed a prognostic and predictive seven-gene assay including ABCC4, CCL19, CD27, DAG1, FUT7, SLC39A8, and ZNF71 for early-stage NSCLC. In this study, we further explored the prognostic capacity of the seven-gene signature using proteomic profiles in a Chinese LUAD cohort from Xu et al [33] (n=103) and TCGA-NSCLC datasets (n=923, TCGA-LUAD and TCGA-LUSC combined) patient samples with sufficient survival information. Immune cell-type activities associated with different prognostic patient groups were investigated.

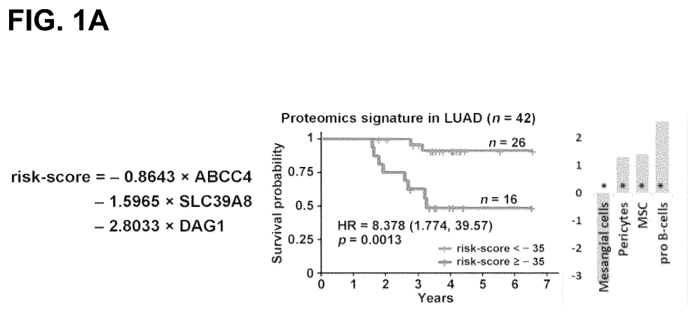

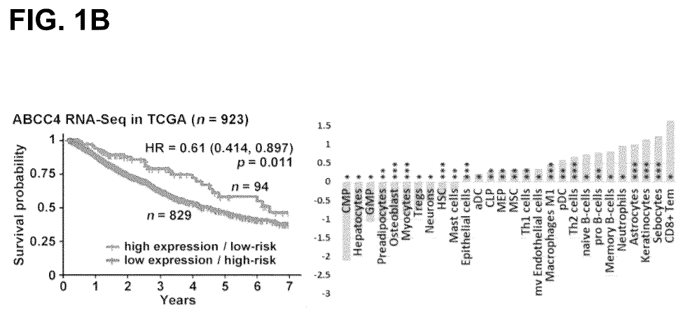

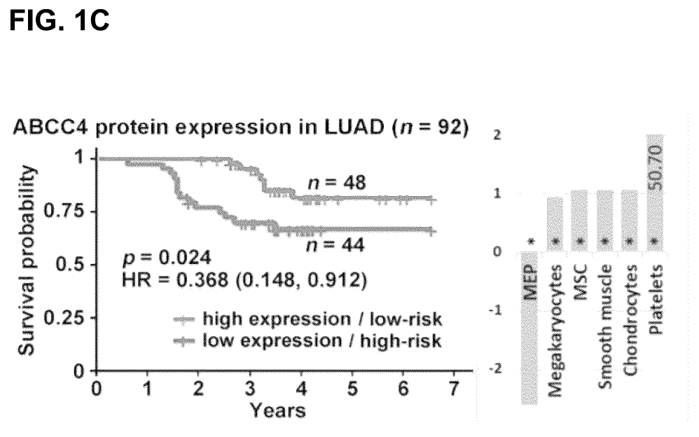

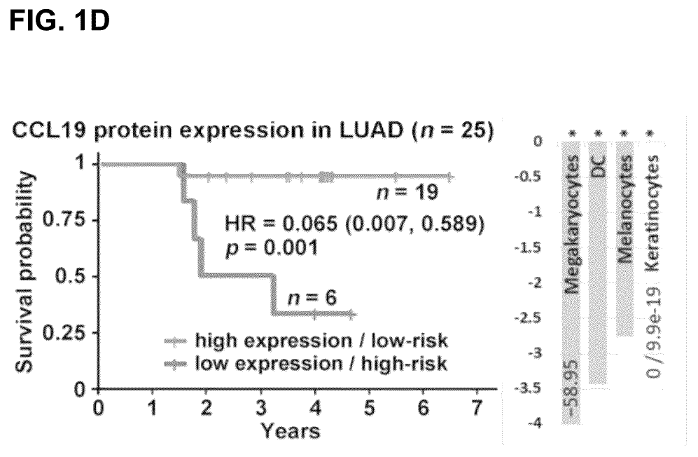

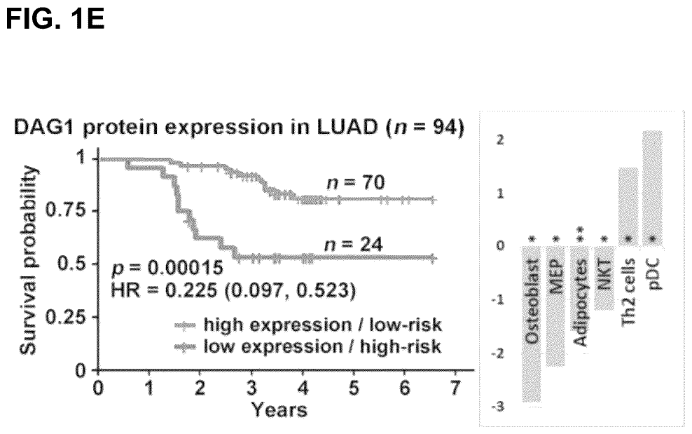

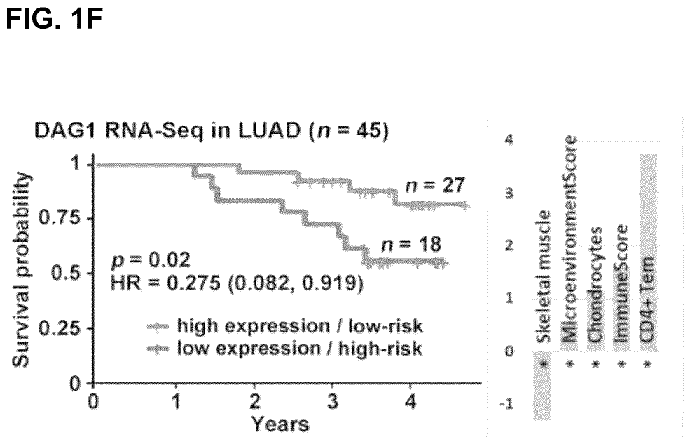

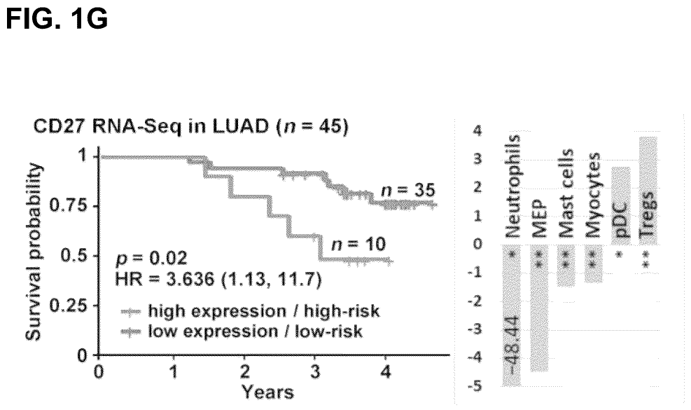

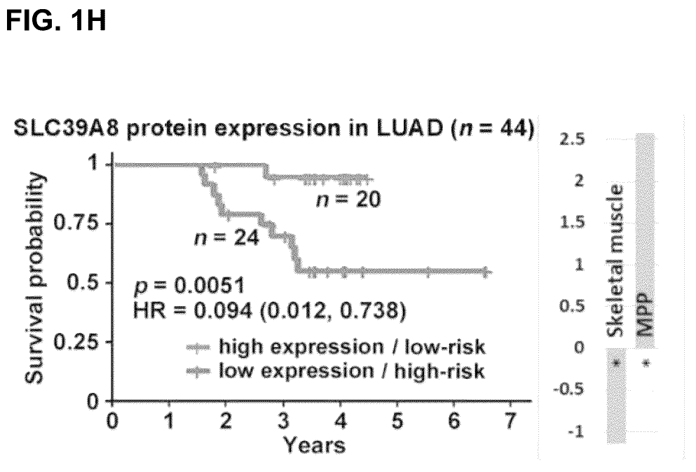

ABCC4, CCL19, CD27, DAG1, and SLC39A8 from the seven-gene assay were available in log₁₀ transformed proteomics data of Xu's LUAD cohort [33]. A multivariate Cox model was built based on these five genes to calculate the coefficients for the risk score. A stepwise model selection that dropped the least significant variable in each iteration was used to reach an optimal model. The final risk-score equation was shown in FIG. 1A. A risk-score cutoff point of −35 was found to stratify the patient samples with significantly different survival outcomes. The Kaplan-Meier analysis results showed that the patients with a risk score lower than −35 had significantly better survival outcomes than the patients with a risk score higher than −35 in Xu's LUAD proteomics data [33] (p=0.0013, HR: 8.378 [1.774, 39.57]; FIG. 1A).

The Kaplan-Meier analysis results also showed significant stratifications for each of these five genes (ABCC4, CCL19, CD27, DAG1, and SLC39A8) in RNA-sequencing/proteomic profiling in Xu's LUAD [33] or TCGA-NSCLC patient cohorts. Patients with a higher expression of ABCC4 (cutoff=10.45) in TCGA-NSCLC RNA-sequencing data survived significantly longer than those with a lower expression of ABCC4 (FIG. 1B). Patients with a higher expression of ABCC4 (cutoff=6.22; FIG. 1C), CCL19 (cutoff=6.31; FIG. 1D), DAG1 (cutoff=6.72; FIG. 1E), and SLC39A8 (cutoff=6.48; FIG. 1H) in log₁₀ transformed proteomics data in Xu's LUAD cohort survived significantly longer than those with a lower protein expression of these genes. respectively. Patients with a higher expression of DAG1 (cutoff=3.9; FIG. 1F) in Xu's LUAD RNA sequencing data [33] survived significantly longer than patients with a lower expression of DAG1. Patients with a higher expression of CD27 (cutoff=8.72; FIG. 1G) in Xu's LUAD RNA sequencing data [33] survived significantly shorter than patients with a lower expression of CD27. ZNF71 and FUT7 did not have any protein expression measurements in Xu's LUAD cohort [33]. In mRNA expression profiles of the TCGA NSCLC and Xu's LUAD [33] cohorts, ZNF71 and FUT7 were not significantly associated with patient survival outcomes.

xCell scores were computed for each patient sample in TCGA-NSCLC and Xu's LUAD [33] cohorts with the corresponding RNA sequencing data. For each significant patient stratification in survival analysis, immune cell types with a significant difference in activities (two-sample t-tests; p<0.05) between high-risk and low-risk patient tumors were identified. The log₂ ratio of xCell scores between high-risk vs. low-risk tumors was shown in FIG. 1a-1h for each stratification, respectively. A positive log₂ (xCell score) indicates that cell type activity varies more in high-risk tumors; a negative log₂ (xCell score) indicates that cell type activity varies more in low-risk tumors.

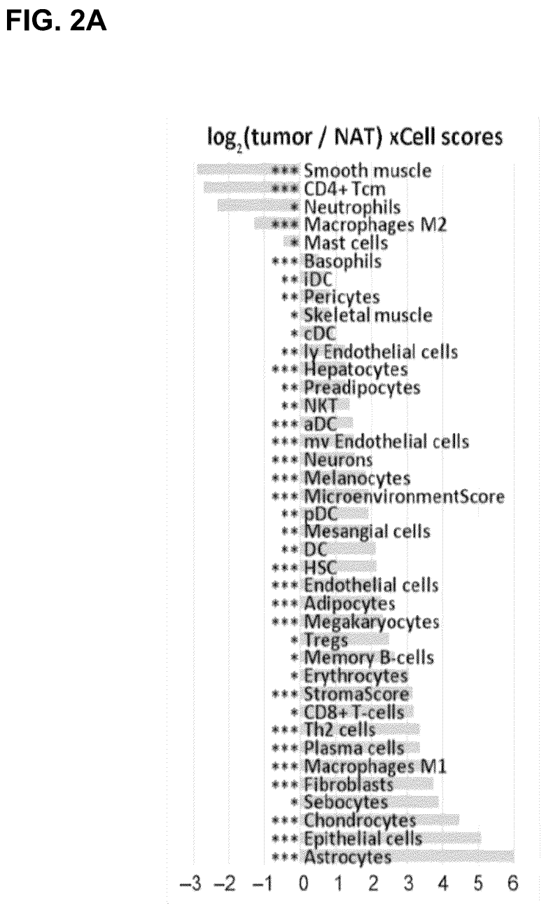

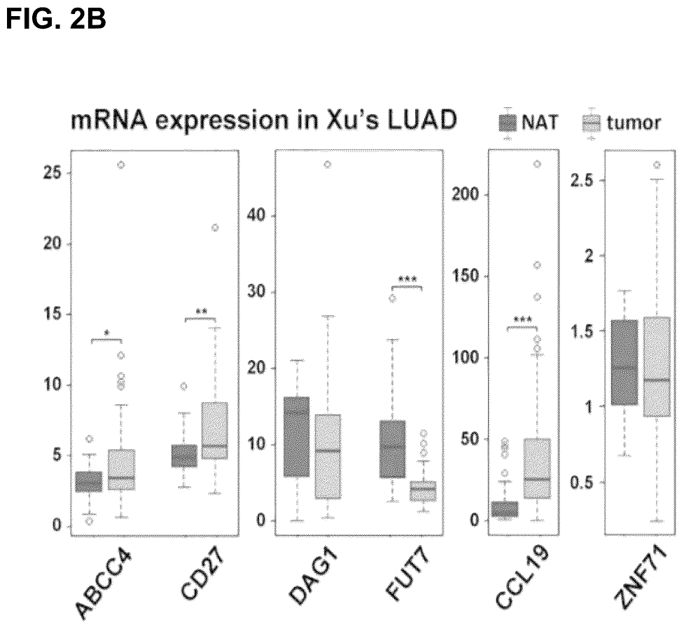

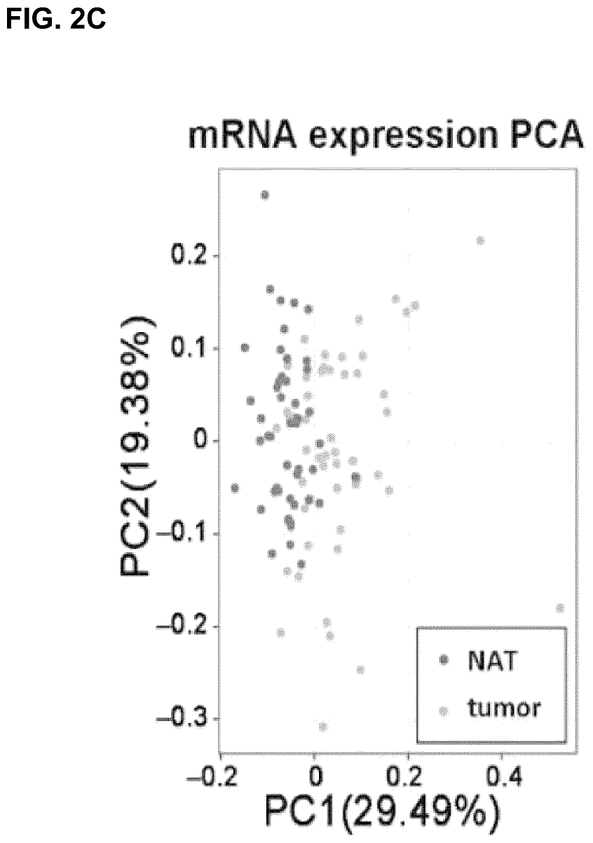

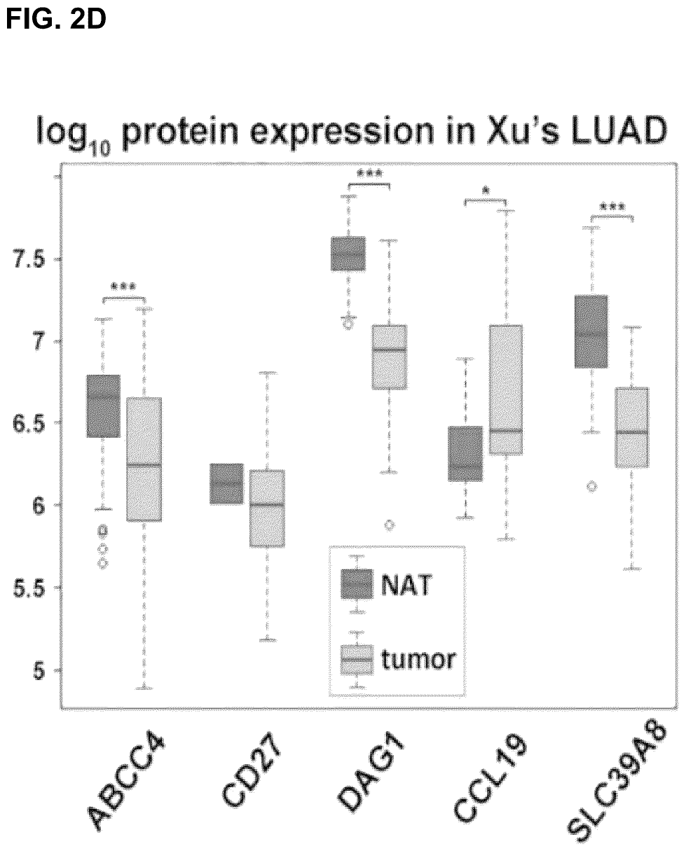

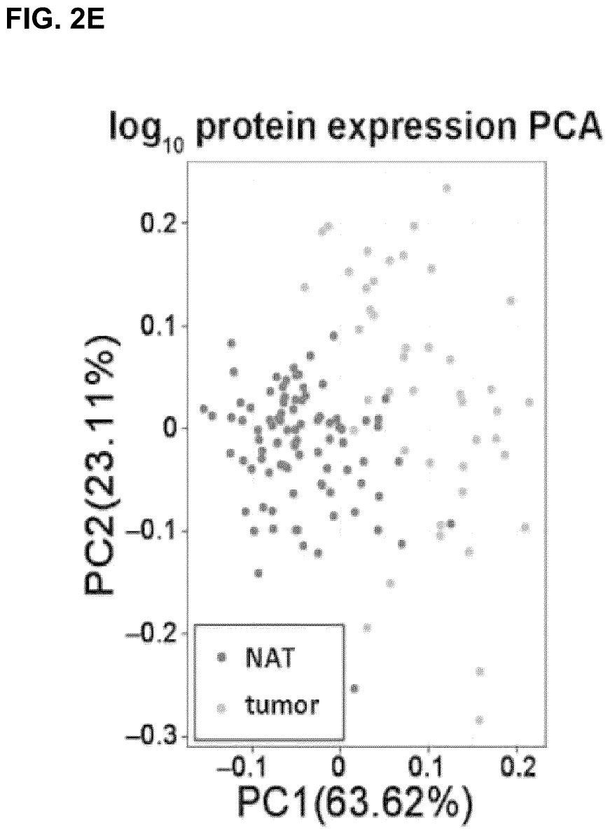

The log₂ ratios of significantly different xCell scores between Xu's LUAD tumors and their paired non-cancerous adjacent tissues (NATs) [33] were also shown in FIG. 2A. The following cell types have more varied levels in NATs than in tumors: smooth muscle, CD4+ central memory T cells (Tcm), neutrophils, macrophages M2, and mast cells. The following cell types have more varied levels in tumors than in NATs: basophils, lentivirus-induced dendritic cells (IDC), pericytes, skeletal muscle, conventional dendritic cells (CDC), ly endothelial cells, hepatocytes, natural killer T cells (NKT), activated dendritic cells (aDC), mv endothelial cells, neurons, melanocytes, microenvironment score (the sum of all immune and stromal cell types), plasmacytoid dendritic cells (pDC), mesangial cells, dendritic cells, hematopoietic stem cells (HSC), endothelial cells, adipocytes, megakaryocytes, Tregs, memory B-cells, erythrocytes, StromaScore (the sum of adipocytes, fibroblasts, and endothelial cells), CD8+ T-cells, Th2 cells, plasma cells, macrophages M1, fibroblasts, sebocytes, chondrocytes, epithelial cells, and astrocytes.

FIG. 1A shows the prognosis of the seven-gene panel using RNA sequencing and proteomic profiles in NSCLC tumors. Kaplan-Meier analysis and log₂ ratio of xCell scores of the high-risk group vs. the low-risk group using three-gene (ABCC4, SLC39A8, and DAG1) cox model in LUAD proteomics data.

FIG. 1B shows the prognosis of the seven-gene panel using RNA sequencing and proteomic profiles in NSCLC tumors. Kaplan-Meier analysis and log₂ ratio of xCell scores of the high-risk group vs. the low-risk group using RNA sequencing data of ABCC4 in TCGA-NSCLC.

FIG. 1C shows the prognosis of the seven-gene panel using RNA sequencing and proteomic profiles in NSCLC tumors. Kaplan-Meier analysis and log₂ ratio of xCell scores of the high-risk group vs. the low-risk group using proteomics data of ABCC4 in LUAD [33].

FIG. 1D shows the prognosis of the seven-gene panel using RNA sequencing and proteomic profiles in NSCLC tumors. Kaplan-Meier analysis and log₂ ratio of xCell scores of the high-risk group vs. the low-risk group using proteomics data of CCL19 in LUAD [33].

FIG. 1E shows the prognosis of the seven-gene panel using RNA sequencing and proteomic profiles in NSCLC tumors. Kaplan-Meier analysis and log₂ ratio of xCell scores of the high-risk group vs. the low-risk group using proteomics data of DAG1 in LUAD [33].

FIG. 1F shows the prognosis of the seven-gene panel using RNA sequencing and proteomic profiles in NSCLC tumors. Kaplan-Meier analysis and log₂ ratio of xCell scores of the high-risk group vs. the low-risk group using RNA sequencing data of DAG1 in LUAD [33].

FIG. 1G shows the prognosis of the seven-gene panel using RNA sequencing and proteomic profiles in NSCLC tumors. Kaplan-Meier analysis and log₂ ratio of xCell scores of the high-risk group vs. the low-risk group using RNA sequencing data of CD27 in LUAD [33].

FIG. 1H shows the prognosis of the seven-gene panel using RNA sequencing and proteomic profiles in NSCLC tumors. Kaplan-Meier analysis and log₂ ratio of xCell scores of the high-risk group vs. the low-risk group using proteomics data of SLC39A8 in LUAD [33].

In FIGS. 1A-H, two sample t-tests were performed to test the difference between the two groups (*: p<0.05; **: p<0.01; ***: p<0.001).

### 2.2. Diagnostic Implication of the Seven-Gene Signature in NSCLC

FIG. 2A shows the classification of non-cancerous adjacent tissues (NATs) and lung adenocarcinoma (LUAD) tumors with the seven-gene panel Log₂ ratio of xCell scores of the LUAD tumor vs. NATs RNA sequencing data. Two sample t-tests were performed to test the difference between the two groups (*: p<0.05; *: p<0.01; *: p<0.001.

FIG. 2B shows the classification of non-cancerous adjacent tissues (NATs) and lung adenocarcinoma (LUAD) tumors with the seven-gene panel boxplots of available RNA sequencing gene expression.

FIG. 2C shows the classification of non-cancerous adjacent tissues (NATs) and lung adenocarcinoma (LUAD) tumors with the seven-gene panel principal component analysis (PCA) of RNA sequencing gene expression.

FIG. 2D shows the classification of non-cancerous adjacent tissues (NATs) and lung adenocarcinoma (LUAD) tumors with the seven-gene panel protein expression in tumors and NATs of the 7-gene panel in LUAD patients [33].

FIG. 2E shows the classification of non-cancerous adjacent tissues (NATs) and lung adenocarcinoma (LUAD) tumors with the seven-gene panel protein expression in separating NATs and LUAD tumors in patients.

We examined the potential of using the seven-gene panel to separate NSCLC tumors from NATs. Within the seven-gene panel, there were six genes (ABCC4, CD27, DAG1, FUT7, CCL19, and ZNF71) available in the RNA sequencing data of Xu's LUAD cohort [33] (FIG. 2B). The principal component analysis (PCA) using the mRNA expression of these six genes to separate tumors and NATs in Xu's LUAD cohort [33] was shown in FIG. 2C. There were five genes (ABCC4, CCL19, CD27, DAG1, and SLC39A8) available in Xu's LUAD proteomics data [33] (FIG. 2D). The separation of LUAD tumors and NATs using these five protein expression data was shown in FIG. 2E. ABCC4 was expressed significantly higher in LUAD tumors than NATs in RNA sequencing data (p<0.05, two sample t-tests, FIG. 2B) but was expressed significantly higher in NATs than in tumors in proteomics data (p<0.001, two sample t-tests, FIG. 2D). CCL19 was expressed significantly higher in LUAD tumors than in NATs in both RNA sequencing and proteomics data (p<0.05, two sample t-tests, FIG. 2B, D). CD27 was expressed significantly higher in LUAD tumors in RNA sequencing data (p<0.01, two sample t-tests, FIG. 2B) but was not significantly different in protein expression between LUAD tumors and NATs (FIG. 2D). DAG1 and SLC39A8 were expressed significantly higher in NATs in LUAD proteomics data (p<0.001, two sample t-tests, FIG. 2D).

To evaluate the accuracy of using the seven-gene panel in classifying tumors from NATs in Xu's LUAD cohort [33], we applied seven commonly used machine-learning classification algorithms. These algorithms included decision tree, k-nearest neighbor (KNN), logistic regression, Naïve Bayes, random forests, Support Vector Machine (SVM), and Radial Basis Function (RBF) network. Classification methods were performed in Weka with 10-fold cross-validation. In LUAD RNA sequencing data [33], six genes (ABCC4, CCL19, CD27, DAG1, FUT7, and ZNF71) with available mRNA expression data were used in classification. Random forests and RBF networks had the highest overall classification accuracy of 0.86. The random forests classification had a sensitivity of 0.882, a specificity of 0.837, a ROC of 0.927, and an odds ratio of 38.44 in the 10-fold cross-validation of tumors vs. NATs (n=51). The RBF network had a sensitivity of 0.824, a specificity of 0.898, a ROC of 0.896, and an odds ratio of 41.07 in the 10-fold cross-validation of tumors vs. NATs. Three genes (ABCC4, DAG1, and SLC39A8) with available protein expression data were used in the classification. SVM had the highest overall classification accuracy of 0.941, with a sensitivity of 0.867, a specificity of 0.978, a ROC of 0.922, and an odds ratio of 286 in classifying LUAD tumors from NATs (n=103). Overall, the 7-gene panel generated high accuracy in separating tumors from NATs using RNA-sequencing or proteomic profiles, indicating its diagnostic implications in NSCLC. Detailed information on the classification results was included in Table 4 and Table 5.

### 2.3. ZNF71 Expression and Selected Immune Cells in NSCLC Prognosis

ZNF71 protein expression was not available in Xu's LUAD cohort [33]. In our previous study, ZNF71 protein expression quantified with AQUA was associated with good prognosis in NSCLC patients (n=191) in tissue microarrays [14]. Although ZNF71 overall mRNA expression was not associated with NSCLC patient survival outcomes, ZNF71 KRAB, the transcriptional repression isoform, was an independent poor prognostic factor in early-stage NSCLC [19]. Furthermore, ZNF71 KRAB was associated with EMT in NSCLC patient tumors (n=197) and epithelial cell lines (n=117). ZNF71 protein expression positively correlated with epithelial markers E-cadherin and Cytokeratin and negatively correlated with mesenchymal markers ZEB1 and Vimentin in Western blots [15], consistent with the association between ZNF71 protein expression and favorable patient prognosis [19]. Based on the structurally relevant function of KRAB-ZNFs, we hypothesized that ZNF71 could be involved in the suppression of endogenous transposable elements (TEs) expression, which is often activated in cancer and can trigger an innate immune response.

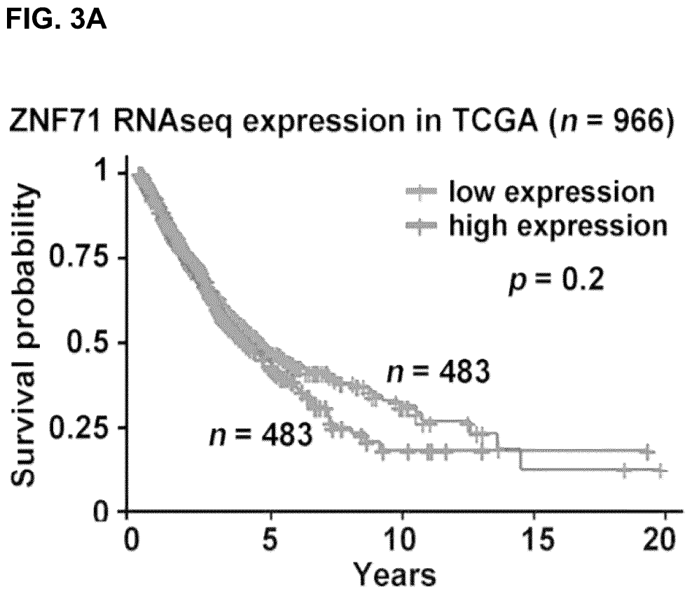

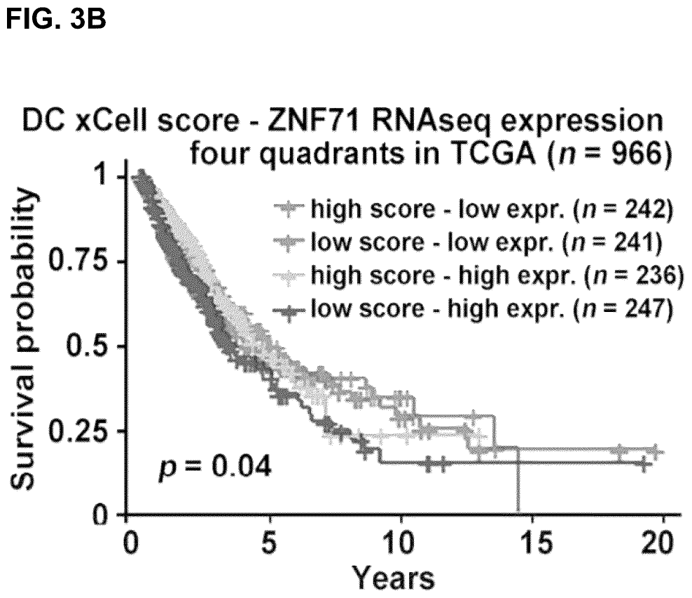

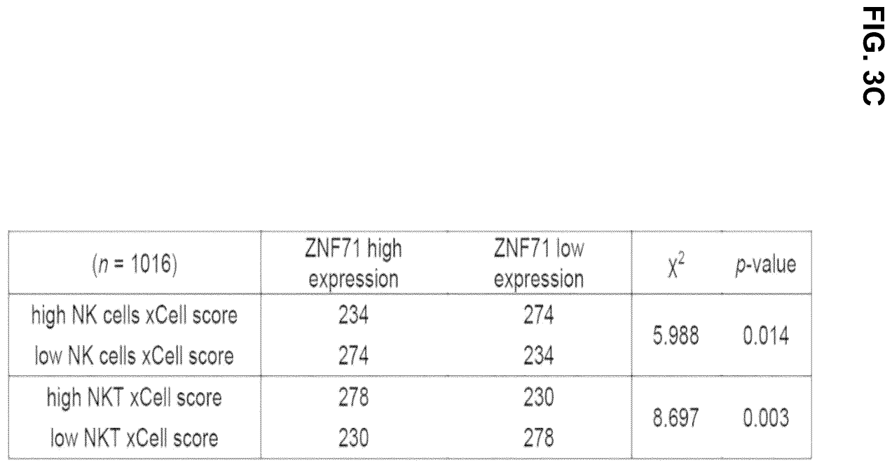

Next, we tested if ZNF71 expression and specific immune cell activities have any associations with NSCLC prognosis. The Kaplan-Meier analysis results showed that if we used the median value of ZNF71 mRNA expression as the cutoff to stratify TCGA-NSCLC, the low-expression group and high-expression group did not have a significant difference in the survival outcomes (FIG. 3A). When we included the Dendritic Cell (DC) xCell score and created a four quadrants stratification with the median of both ZNF71 mRNA expression and DC xCell score, i.e. high DC xCell score−low ZNF71 expression, low DC xCell score−low ZNF71 expression, high DC xCell score−high ZNF71 expression, and low DC xCell score−high ZNF71 expression, the Kaplan-Meier analysis result showed a significant difference in survival among the four groups (log-rank p=0.04, FIG. 3B). These results showed that patients with different ZNF71 expressions and DC activities had distinct survival outcomes. Those with high DC xCell scores (representing more varied DC levels) and low ZNF71 expression had the best survival outcomes; whereas those with low DC xCell scores (representing less varied DC levels) and high ZNF71 expression had the worse survival outcomes (FIG. 3B). Out of all available cell types analyzed with xCell, the DC xCell score is the only metric that can generate significant prognostic stratification when combined with ZNF71 gene expression in TCGA NSCLC patients. Furthermore, ZNF71 expression level had significant associations with the xCell scores of natural killer (NK) cells and NKT in TCGA NSCLC patient tumors (p<0.01, χ² tests, FIG. 3C). These results are consistent with a proposed model that tumor-derived substances trigger the early generation of IFN-β by host CD11c+ DCs. The cross-presentation of tumor-derived antigens is then stimulated by this IFN-β acting on the CD8α+ DC subset, resulting in the cross-priming of CD8+ T cells specific for the tumor antigen. These reactivated T lymphocytes may then move toward the tumor and cause more tumor cell death [34].

FIG. 3A shows the association of ZNF71 and xCell scores of selected immune cells. Prognostic implications of ZNF71 and xCell scores of dendritic cells in TCGA NSCLC patient tumors. Kaplan-Meier analysis of TCGA-NSCLC patients stratified by the median value of ZNF71 mRNA expression.

FIG. 3B shows the association of ZNF71 and xCell scores of selected immune cells. Prognostic implications of ZNF71 and xCell scores of dendritic cells in TCGA NSCLC patient tumors. Kaplan-Meier analysis of TCGA-NSCLC patients stratified by the median value of ZNF71 mRNA expression and the median value of dendritic cells (DC) xCell scores.

FIG. 3C shows the association of ZNF71 and xCell scores of selected immune cells. Prognostic implications of ZNF71 and xCell scores of dendritic cells in TCGA NSCLC patient tumors. χ² test results of ZNF71 expression vs. xCell scores of NK cells and NKT in TCGA-NSCLC patient tumors.

### 2.4. Overexpression of ZNF71 KRAB, KRAB-Less Isoforms Suppresses Innate Immune Response

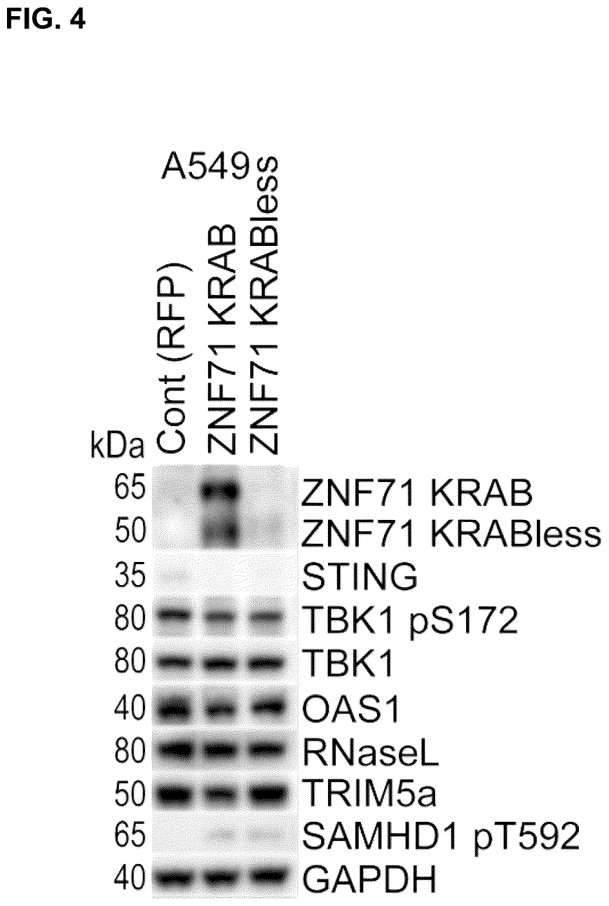

To investigate the potential function of ZNF71 in intracellular immune response, we overexpressed its KRAB and KRABless isoforms in lung adenocarcinoma A549 cells (FIG. 4). The latter is missing the KRAB domain (as a result of spliced-out exon 3) and encodes an approximately 55 kDa protein. Interestingly, ZNF71 overexpression led to the downregulation of STING and its downstream effector S172-phosphorylated TBK1, while the total TBK1 level was not decreased. Similarly, we observed downregulation of OAS1, while RNase L expression was not significantly changed. Two viral restriction factors were either downregulated, TRIM5a, or inactivated by phosphorylation, SAMHD1 pT592 (FIG. 4). These data suggest that overexpression of ZNF71 results in decreased expression of multiple components of the intracellular intrinsic and innate immune systems, including dsRNA and dsDNA sensors. Although direct targets of ZNF71 have not been identified yet, these results are consistent with a hypothesis that ZNF71 suppresses the transcription of genomic TEs.

FIG. 4 shows that ZNF71 suppresses components of the innate and intrinsic immune response. Western blotting of control RFP, ZNF71 KRAB, and KRABless isoforms overexpression, as well as of several markers of intracellular innate and intrinsic immune systems in A549 cells. GAPDH is shown as the loading control.

### 2.5. Gene Association Networks of ZNF71 and Intracellular Innate Response Genes

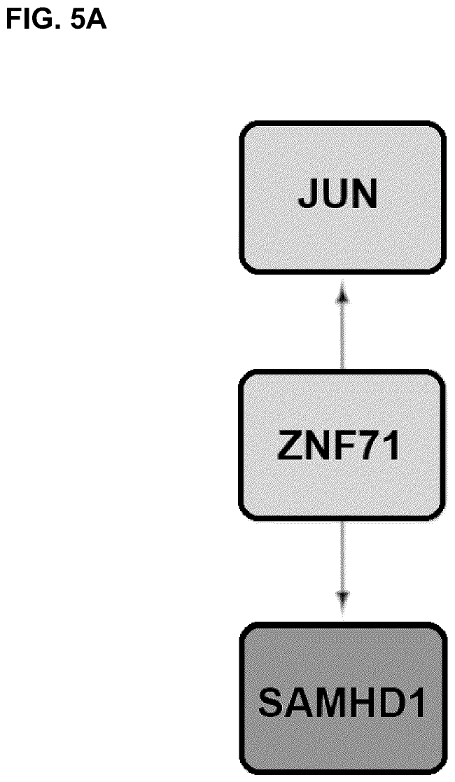

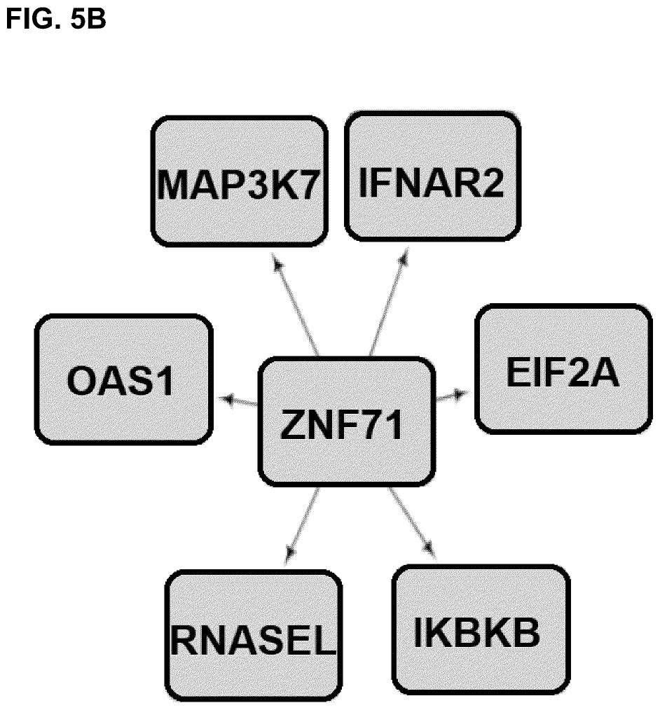

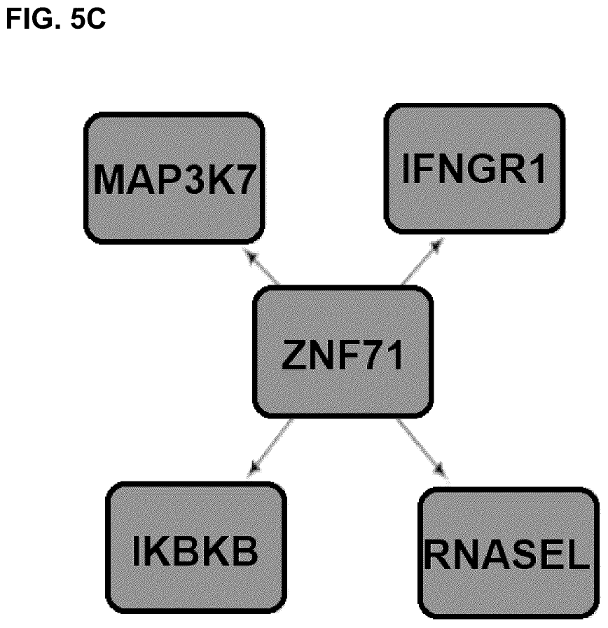

To explore gene interactions and pathways among ZNF71 and the intracellular intrinsic and innate immune systems, multi-omics association networks involving ZNF71 and genes examined in Western blot (FIG. 4) were computed with the Boolean implication network method in Xu's LUAD cohort [33] containing both mRNA and protein expression profiles in tumors and NATs. The intracellular innate immune response (IIIR) genes included (1) interferons and their receptors: IFNA16, IFNA17, IFNA21, IFNA22P, IFNA4, IFNA5, IFNAR1, IFNAR2, IFNE, IFNG, IFNG-AS1, IFNGR1, IFNGR2, IFNK, IFNL1, IFNL3, IFNLR1, IFNW1; (2) cGAS-STING pathway: CGAS, TMEM173/STING1, TBK1, IKBKB, IRF3, IRF7, AIM2, maybe JUN, and MAP3K7; (3) OAS-RNase L pathway: OAS1 and RNASEL; (4) viral restriction factors: SAMHD1 and TRIM5; (5) cyclin-dependent kinase: CDK1; (6) co-repressor for KRAB-ZNFs: TRIM28; and (7) housekeeping glycolysis gene: PFKL. For the seven-gene panel and the IIIR genes, the status of proliferation as measured in CRISPR-Cas9/RNAi in NSCLC cell lines and differential expression analysis in each studied patient cohort was included in Table 6. First, direct mRNA co-expression networks of ZNF71 and IIIR genes (p<0.05, z tests) were found when (1) ZNF71 was up-regulated in LUAD tumors (FIG. 5A), (2) ZNF71 was up-regulated in NATs (FIG. 5B), and (3) ZNF71 was down-regulated in NATs (FIG. 5C) in the analysis of RNA-sequencing data from Xu et al [33]. No significant direct gene co-expression relations were found between ZNF71 and IIIR genes when ZNF71 was downregulated in LUAD tumors.

FIG. 5A shows direct gene co-expression networks of ZNF71 and intracellular innate immune response (IIIR) genes in lung adenocarcinoma (LUAD) patient samples using RNA sequencing data. Direct gene associations of ZNF71 and IIIR genes when ZNF71 is upregulated in LUAD tumors.

FIG. 5B shows direct gene co-expression networks of ZNF71 and intracellular innate immune response (IIIR) genes in lung adenocarcinoma (LUAD) patient samples using RNA sequencing data. Direct gene associations of ZNF71 and IIIR genes when ZNF71 is upregulated in NATs.

FIG. 5C shows direct gene co-expression networks of ZNF71 and intracellular innate immune response (IIIR) genes in lung adenocarcinoma (LUAD) patient samples using RNA sequencing data. Direct gene associations of ZNF71 and IIIR genes when ZNF71 is downregulated in NATs.

The computed direct gene associations between ZNF71 and IIIR genes (FIG. 5A-C) did not provide sufficient information to infer signaling pathways relevant to ZNF71-mediated innate immune responses. Here, the computationally derived gene associations do not represent biological interactions one would expect to observe in genome-scale profiling after ZNF71 overexpression/knockdown. To infer pathways and interactions between ZNF71 and IIIR genes, we expanded the gene association networks as follows.

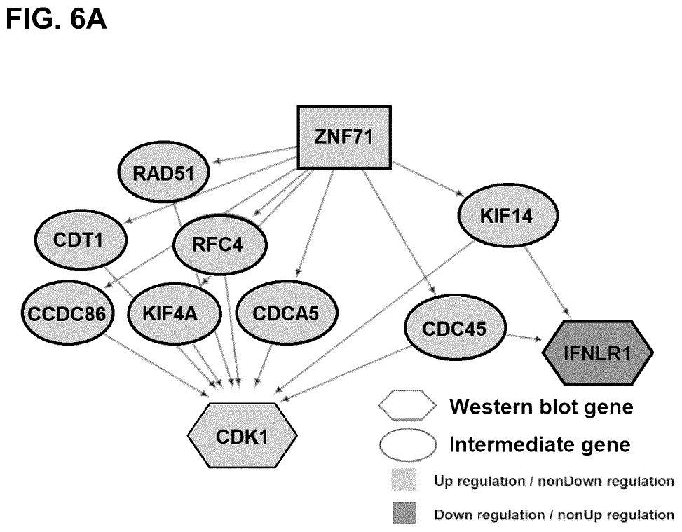

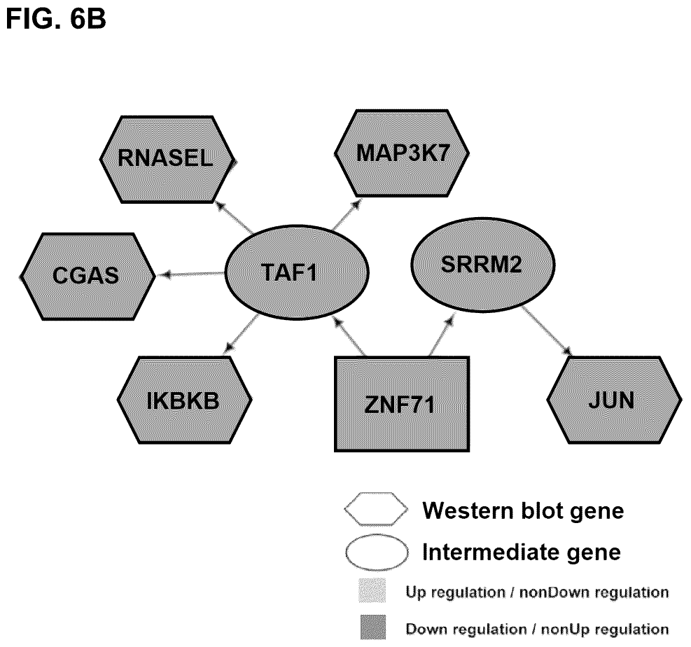

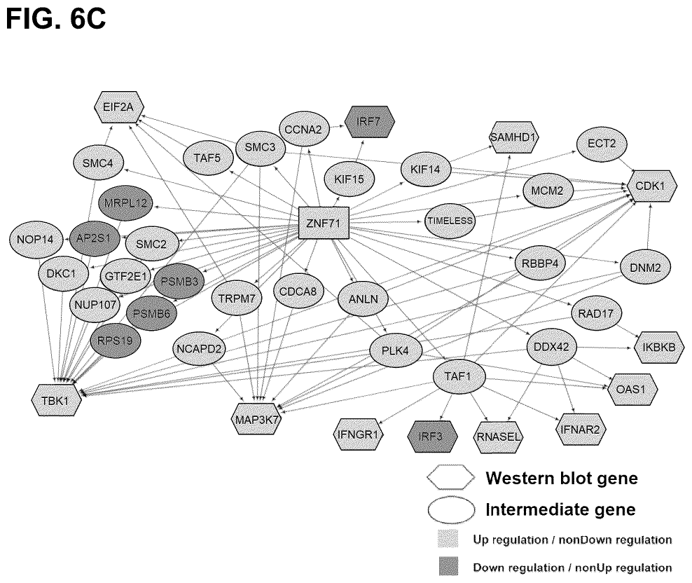

Second, we also identified indirect gene association networks between ZNF71 and IIIR genes using RNA sequencing data from Xu et al [33]. ZNF71 had co-expression associations with IIIR genes (p<0.05, z tests) through some intermediate genes. To form a manageable size of networks, we filtered the intermediate genes with the following criteria: (1) the gene was differentially expressed between LUAD tumors vs. NATs (p<0.05, two-sample t-tests); (2) the gene was a proliferation gene that had a significant effect (dependency score<−0.5) on 50% or more NSCLC cell lines in RNAi or CRISPR-Cas9 screening data; (3) the gene was a prognostic gene that can significantly stratify the patient survival in RNA sequencing data of both TCGA-LUAD and Xu's LUAD [33] patient cohorts. The indirect gene association networks with intermediate genes that meet all three criteria were found when ZNF71 was up-regulated in LUAD tumors (FIG. 6A), when ZNF71 was down-regulated in NATs (FIG. 6B), and when ZNF71 was up-regulated in NATs (FIG. 6C). Again, no significant indirect gene co-expression relations were found between ZNF71 and IIIR genes when ZNF71 was downregulated in LUAD tumors.

FIG. 6A shows indirect gene co-expression networks of ZNF71 and intracellular innate immune response (IIIR) genes in lung adenocarcinoma (LUAD) patient samples using RNA sequencing data. Gene associations of ZNF71 with IIIR genes through intermediate genes in LUAD tumors when ZNF71 is upregulated.

FIG. 6B shows indirect gene co-expression networks of ZNF71 and intracellular innate immune response (IIIR) genes in lung adenocarcinoma (LUAD) patient samples using RNA sequencing data. Gene associations of ZNF71 with IIIR genes through intermediate genes in NATs when ZNF71 is downregulated.

FIG. 6C shows indirect gene co-expression networks of ZNF71 and intracellular innate immune response (IIIR) genes in lung adenocarcinoma (LUAD) patient samples using RNA sequencing data. Gene associations of ZNF71 with IIIR genes through intermediate genes in NATs when ZNF71 is upregulated.

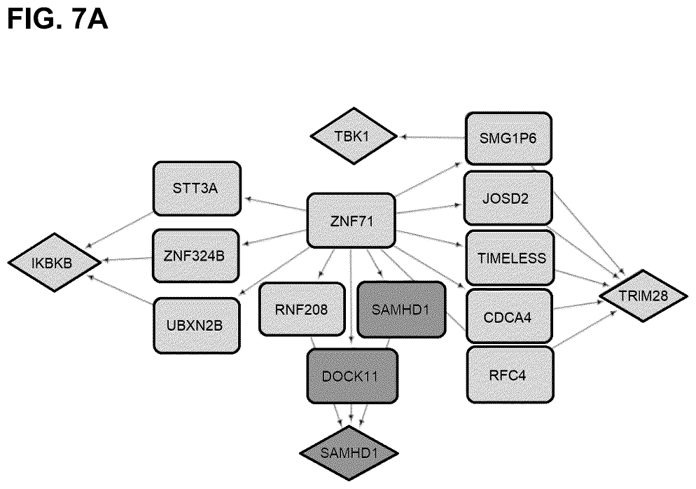

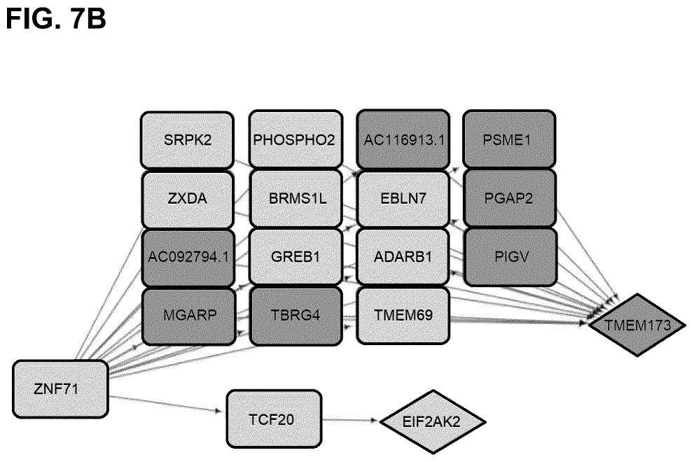

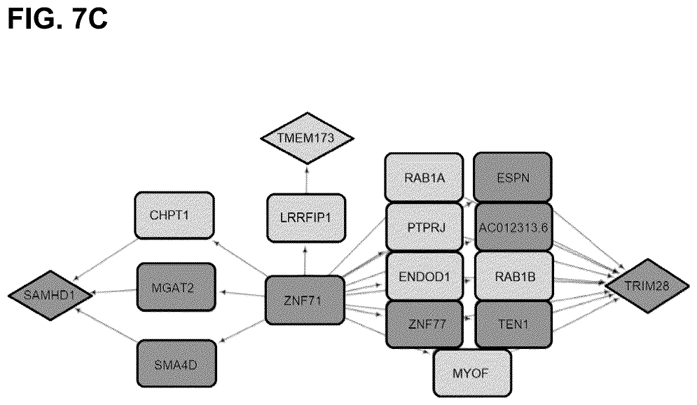

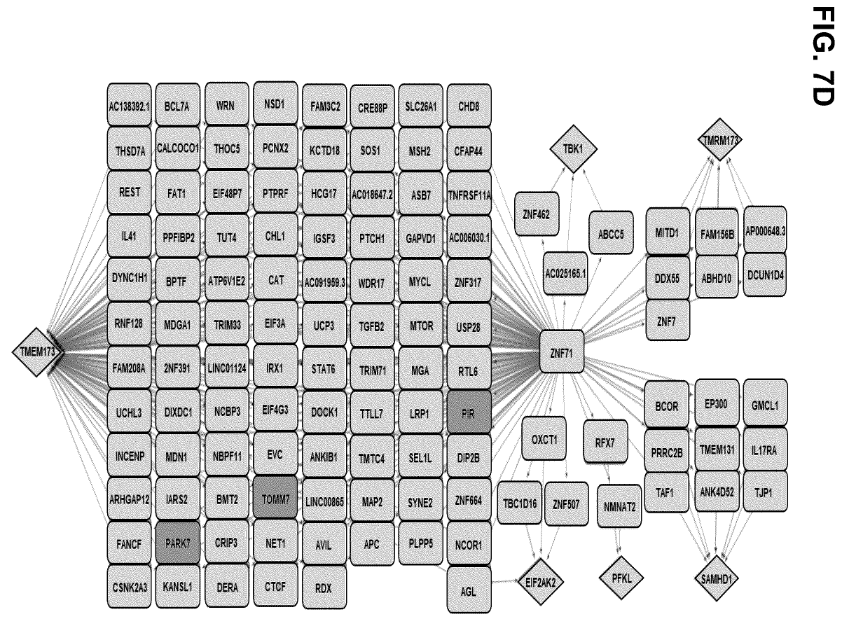

FIG. 7A shows multi-omics gene association networks of ZNF71 and intracellular innate immune response (IIIR) genes in lung adenocarcinoma (LUAD) patient samples using RNA sequencing and proteomics data. Gene associations between ZNF71 and IIIR genes through intermediate genes in LUAD tumors when ZNF71 is upregulated.

FIG. 7B shows multi-omics gene association networks of ZNF71 and intracellular innate immune response (IIIR) genes in lung adenocarcinoma (LUAD) patient samples using RNA sequencing and proteomics data. Gene associations between ZNF71 and IIIR genes through intermediate genes in NATs when ZNF71 is upregulated.

FIG. 7C shows multi-omics gene association networks of ZNF71 and intracellular innate immune response (IIIR) genes in lung adenocarcinoma (LUAD) patient samples using RNA sequencing and proteomics data. Gene associations between ZNF71 and IIIR genes through intermediate genes in LUAD tumors when ZNF71 is downregulated.

FIG. 7D shows multi-omics gene association networks of ZNF71 and intracellular innate immune response (IIIR) genes in lung adenocarcinoma (LUAD) patient samples using RNA sequencing and proteomics data. Gene associations between ZNF71 and IIIR genes through intermediate genes in NATs when ZNF71 is downregulated.

Third, although ZNF71 was not available in LUAD proteomics data from Xu et al [33], ZNF71 mRNA expression had indirect associations with the protein expression of some IIIR genes through intermediate genes. The indirect gene association networks of ZNF71 (mRNA expression)->intermediate genes (mRNA expression)->IIIR genes (protein expression) were found in LUAD tumors when ZNF71 was up-regulated (FIG. 7A) and when ZNF71 was down-regulated (FIG. 7C), in NATs when ZNF71 was up-regulated (FIG. 7B) and when ZNF71 was down-regulated (FIG. 7D).

We examined the pathways in ZNF71 co-expression networks with ToppGene. Genome-scale ZNF71 co-expression networks containing all the genes with a significant association (p<0.05, z tests) with ZNF71 were constructed with the Boolean implication networks using RNA sequencing data in LUAD tumors and NATs from Xu et al [33], respectively. For each disease state, the significantly (p<0.05) enriched pathways of the gene co-expression networks when ZNF71 was upregulated or downregulated were compared. There were 30 common pathways between the networks when ZNF71 was upregulated or downregulated in NATs, focusing on DNA repair, RTK signaling, and transcriptional regulation. There were no common pathways between the networks when ZNF71 was upregulated or downregulated in LUAD tumors. Of the 30 common pathways in NATs, generic transcription pathway (ToppGene ID: 1269650) and gene expression (ToppGene ID: 1269649) were present in the pathways when ZNF71 was upregulated in LAUD tumors; whereas membrane trafficking (ToppGene ID: 1269877) and vesicle-mediated transport (ToppGene ID: 1269876) showed up in the pathways associated with ZNF71 downregulated network in LUAD tumors. Once we focused the gene co-expression networks on ZNF71 and IIIR genes when ZNF71 was upregulated or downregulated, 21 common pathways relevant to immune response were found in LUAD tumors; two common pathways, gene expression and generic transcription pathway, were found in NATs. Detailed significantly (p<0.05) enriched pathways associated with ZNF71 genome-wide co-expression networks, indirect networks of ZNF71 and IIIR genes without any filtering criteria applied, and networks shown in FIGS. 6A-C and FIGS. 7A-D.

### 2.6. Identification of Genes Associated With Drug Response

A total of 21 NCCN-recommended drugs for systemic or targeted therapy for treating NSCLC were available in the Cancer Cell Line Encyclopedia (CCLE) drug screening data. We sought to identify pan-sensitive and pan-resistant genes to these 21 drugs using CCLE RNA sequencing and proteomics profiles in human NSCLC cell lines. The following genes were included in the drug-sensitivity analysis: 1) the seven-gene panel, 2) selected epithelial genes (CDH1, EPCAM, ESRP1, ESRP2, DDR1, CTNNB1, CD24, CLDN7, KRT8, KRT19, and RAB25) and mesenchymal genes (ZEB1, VIM, and FN1), 3) IIIR genes (FIG. 4), and 4) all the genes in the ZNF71-IIIR gene association networks (FIGS. 5A-C and FIG. 7A-D). Genes that were expressed significantly higher (p<0.05; two-sample t-tests) in sensitive NSCLC cell lines for a specific drug were defined as sensitive genes. The epithelial and mesenchymal genes were included because the ZNF71 KRAB isoform was associated with EMT, and a 14-gene EMT classifier containing these genes separated early-stage NSCLC patients into distinct prognostic groups with disparate survival outcomes [19]. For a specific drug, genes that were expressed significantly higher (p<0.05; two-sample t-tests) in resistant NSCLC cell lines were defined as resistant genes. In this study, we only selected the genes that were pan-sensitive or pan-resistant (Table 1 and Table 2). Pan-sensitive genes were the genes that were identified as either sensitive or not resistant to all the studied 21 drugs. Similarly, pan-resistant genes were the genes that were identified as either resistant or not sensitive to all the studied 21 drugs. PRISM [35] and GDSC1/2 [36-38] drug screening data were included in this analysis.

TABLE 1
Pan-sensitive and pan-resistant genes to 21 drugs using RNA sequencing data in CCLE NSCLC cell lines (n = 135).

| drug | systemic/targeted therapy | Pan-sensitive genes | Pan-resistant genes |
|---|---|---|---|
| afatinib | EGFR Exon 19 Deletion or L858R/ | CDT1, INCENP | IL4I1, LRP1, FAM156B, |
| | EGFR S768I, L861Q, and/or | | DCUN1D4, ESPN, PTPRJ, |
| | G719X | | NMNAT2 |
| alectinib | ALK Rearrangement Positive | CSNK2A3, DYNC1H1, PSME1 | UCP3, CDC45, THSD7A, |
| | | | ANKRD52, EIF2A, FAM156B, |
| | | | KRT8, CFAP44 |
| brigatinib | ALK Rearrangement Positive | | MYOF, ABHD10, TMTC4 |
| cabozantinib | RET Rearrangement Positive | CSNK2A3 | RNASEL, TMTC4, SMC2, ASB7, |
| | | | CFAP44, IL17RA, ZXDA, |
| | | | THSD7A |
| carboplatin | Systemic | | ASB7, RAB1A |
| cisplatin | Systemic | ZNF507, CSNK2A3, WDR17 | ARHGAP12, CDC45, ESPN, |
| | | | EIF2A, IL17RA, MYOF, TMTC4, |
| | | | NMNAT2, PTPRJ, KRT8 |
| crizotinib | ALK Rearrangement Positive/ | RFC4, CSNK2A3, MCM2, MDN1, | LRP1, IL17RA |
| | ROS1 Rearrangement Positive/ | CDT1, INCENP | |
| | MET Exon 14 Skipping Mutation | | |
| dabrafenib | BRAF V600E Mutation Positive | FBLN7, MAP2, CTNNB1, RNF128, | FAM156B, SLC39A8, SMC2, |
| | | CSNK2A3, WDR17, ZNF507 | KRT8, NMNAT2 |
| dacomitinib | EGFR Exon 19 Deletion or L858R/ | CDT1 | MAP3K7, DCUN1D4, LRP1, |
| | EGFR S768I, L861Q, and/or | | THOC5, FAM156B, ANKRD52, |
| | G719X | | THSD7A |
| docetaxel | Systemic | RFC4, ABCC5, FBLN7, MAP2 | SLC39A8, IL17RA, KRT8, RAB1A, |
| | | | THSD7A, DCUN1D4, MYOF, |
| | | | NMNAT2 |
| erlotinib | EGFR Exon 19 Deletion or L858R/ | CSNK2A3, PSMB6, TGFB2 | FAM156B, ASB7, TMTC4, |
| | EGFR S768I, L861Q, and/or | | NMNAT2 |
| | G719X | | |
| etoposide | Systemic | PSMB3, CDT1, DYNC1H1, ZNF507 | ESPN, NMNAT2, ZNF324B, |
| | | | IL17RA |
| gefitinib | EGFR Exon 19 Deletion or L858R/ | BRMS1L, CCNA2, CDT1, INCENP | MAP3K7, DCUN1D4, NMNAT2, |
| | EGFR S768I, L861Q, and/or | | IL4I1, ABHD10, FAM156B, |
| | G719X | | PTPRJ, KRT8 |
| gemcitabine | Systemic | MDGA1, MDN1, ZNF507, WDR17 | KRT8, ANKRD52, ASB7, RAB1A, |
| | | | FAM156B, IL17RA, DCUN1D4, |
| | | | IL4I1, LRP1, NMNAT2 |
| lorlatinib | ALK Rearrangement Positive/ | MDN1, PSMB6 | ANKRD52, ESPN, RAB1A |
| | ROS1 Rearrangement Positive | | |
| osimertinib | EGFR Exon 19 Deletion or L858R/ | ABCC5, MDGA1, TCF20, MAP2, | ASB7, DCUN1D4, THSD7A |
| | EGFR S768I, L861Q, and/or | PSMB6 | |
| | G719X | | |
| paclitaxel | Systemic | INCENP, CDT1, MCM2, TCF20 | RAB1A, THOC5, ASB7, IL17RA, |
| | | | NMNAT2, KRT8, LRP1, SLC39A8, |
| | | | THSD7A, UCP3 |
| pemetrexed | Systemic | MCM2, CCNA2 | EIF2A, IL17RA, MYOF, THSD7A, |
| | | | ZXDA, ARHGAP12, LRP1 |
| trametinib | BRAF V600E Mutation Positive | CTNNB1 | RNASEL, IL17RA, ANKRD52, |
| | | | FAM156B, AP2S1, ARHGAP12, |
| | | | SRRM2, UCP3, ZXDA, TMTC4, |
| | | | DCUN1D4, NCOR1, RAB1A |
| vemurafenib | BRAF V600E Mutation Positive | | RNASE L, IL17RA |
| vinorelbine | Systemic | ABCC5, DYNC1H1, IRX1, TCF20 | RAB1A, SLC39A8, KRT8, |
| | | | DCUN1D4, PTPRJ, IL17RA, |
| | | | MYOF, NMNAT2 |

TABLE 2
Pan-sensitive and pan-resistant genes to 21 drugs using proteomics data in CCLE NSCLC cell lines (n = 64).

| drug | systemic/targeted therapy | Pan-sensitive genes | Pan-resistant genes |
|---|---|---|---|
| afatinib | EGFR Exon 19 Deletion or L858R/ | DDX42, RFX7, FAT1, CREBBP | IL4I1, KRT8, CDCA4, DIXDC1 |
| | EGFR S768I, L861Q, and/or | | |
| | G719X | | |
| alectinib | ALK Rearrangement Positive | DDX42, ZNF507 | CCNA2, KIF14, CDK1, DAG1, |
| | | | ANKRD52, MAP3K7 |
| brigatinib | ALK Rearrangement Positive | FAT1, PTPRJ, PSME1 | TEN1, MAP3K7, FBLN7, CDK1 |
| cabozantinib | RET Rearrangement Positive | KANSL1, CHL1, TGFB2 | TJP1_G3V1L9, BPTF_E9PE19, |
| | | | NSD1, SYNE2_Q8WXH0_5, |
| | | | RDX, ANKRD52 |
| carboplatin | Systemic | | TJP1_G3V1L9 |
| cisplatin | Systemic | MYCL, DDX42, BRMS1L, KANSL1, | BPTF_E9PE19, FBLN7, DIP2B, |
| | | ZNF507 | MYOF, CDK1 |
| crizotinib | ALK Rearrangement Positive/ | FAM208A, TOMM7, KANSL1 | DAG1 |
| | ROS1 Rearrangement Positive/ | | |
| | MET Exon 14 Skipping Mutation | | |
| dabrafenib | BRAF V600E Mutation Positive | BRMS1L, RBBP4, ZNF507, DDX42, | RDX, ANKRD52, DIP2B, CDK1, |
| | | KANSL1 | DAG1, IL17RA |
| dacomitinib | EGFR Exon 19 Deletion or L858R/ | PSME1, DDX42, GREB1, RBBP4 | MAP2_P11137_4, KRT8, CDC45, |
| | EGFR S768I, L861Q, and/or | | CDCA4, IL4I1 |
| | G719X | | |
| docetaxel | Systemic | TOMM7, CREBBP, TCF20, DDX42, | DYNC1H1, MYOF |
| | | FAM208A, THOC5, ZNF507 | |
| erlotinib | EGFR Exon 19 Deletion or L858R/ | RFX7, THOC5, CREBBP | KIF14, CDCA4 |
| | EGFR S768I, L861Q, and/or | | |
| | G719X | | |
| etoposide | Systemic | CHL1, FAT1 | |
| gefitinib | EGFR Exon 19 Deletion or L858R/ | DDX42, CREBBP, TCF20, CHL1 | CDCA4, IL4I1 |
| | EGFR S768I, L861Q, and/or | | |
| | G719X | | |
| gemcitabine | Systemic | DDX42, CREBBP, ZNF507, RBBP4, | DIP2B, ANKRD52, CDK1, |
| | | TOMM7 | MAP2_P11137_4, IL17RA, |
| | | | SLC39A8 |
| lorlatinib | ALK Rearrangement Positive/ | ASB7, PSME1, PIR | CCNA2, MYOF, CDK1, CDC45 |
| | ROS1 Rearrangement Positive | | |
| osimertinib | EGFR Exon 19 Deletion or L858R/ | | CDCA4, EIF4G3_B1AN89, IL4I1, |
| | EGFR S768I, L861Q, and/or | | ADARB1, CDC45 |
| | G719X | | |
| paclitaxel | Systemic | CHL1, THOC5, ZNF507, UCHL3 | SLC39A8, DIXDC1, DIP2B, |
| | | | MYOF, IL17RA |
| pemetrexed | Systemic | THOC5 | BPTF_E9PE19, RDX, DYNC1H1 |
| trametinib | BRAF V600E Mutation Positive | CHL1, PSME1, TMTC4, JOSD2, | RDX, DAG1, IL17RA, MAP3K7 |
| | | PTPRJ, RPS19 | |
| vemurafenib | BRAF V600E Mutation Positive | FAM208A | KRT8 |
| vinorelbine | Systemic | TCF20, UCHL3, RBBP4, THOC5, | TJP1_G3V1L9 |
| | | ZNF507 | |

### 2.7. Functional Pathways Associated With the ZNF71 Co-Expression Networks and Discovery of Therapeutic Targets

To discover functional pathways and therapeutic targets to improve NSCLC treatment outcomes, pan-sensitive (n=23) and pan-resistant (n=31) genes in RNA sequencing data (Table 1) were used as CMap input candidate genes. The up-regulation of pan-sensitive genes and down-regulation of pan-resistant genes are expected to enhance NSCLC treatment response. Thus, the pan-sensitive genes and the pan-resistant genes were used as the initial up- and down-regulated gene lists in the CMap, respectively. The following steps were further applied to the gene lists to inhibit NSCLC proliferation, reverse EMT, and induce immune response: (1) excluding proliferation genes that had a significant effect (dependency score<−0.5) on at least 50% NSCLC cell lines in both CRISPR-Cas9 and RNAi from the up-regulated gene list; (2) excluding survival protective genes (p<0.05, HR<1; univariate cox-model in RNA sequencing data of Xu's LUAD cohort [33] and TCGA-NSCLC data) from the down-regulated gene list; (3) excluding the hazard genes (p<0.05, HR>1; univariate cox-model in RNA sequencing data of Xu's LUAD cohort [33] and TCGA-NSCLC data) from the up-regulated gene list; (4) excluding the mesenchymal genes from the up-regulated gene list; (5) excluding epithelial genes from the down-regulated gene list; (6) adding CD27, PD1 (PDCD1), and PDL1 (CD274) in the downregulated gene list.

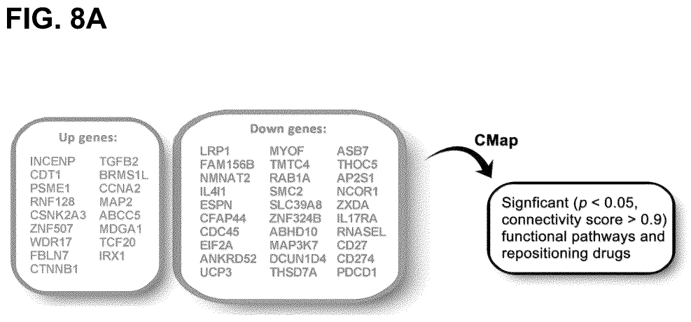

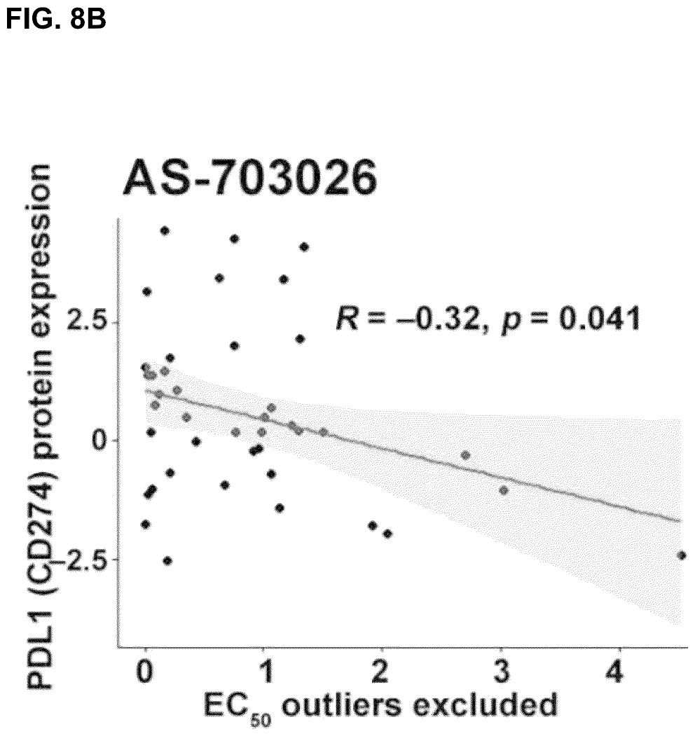

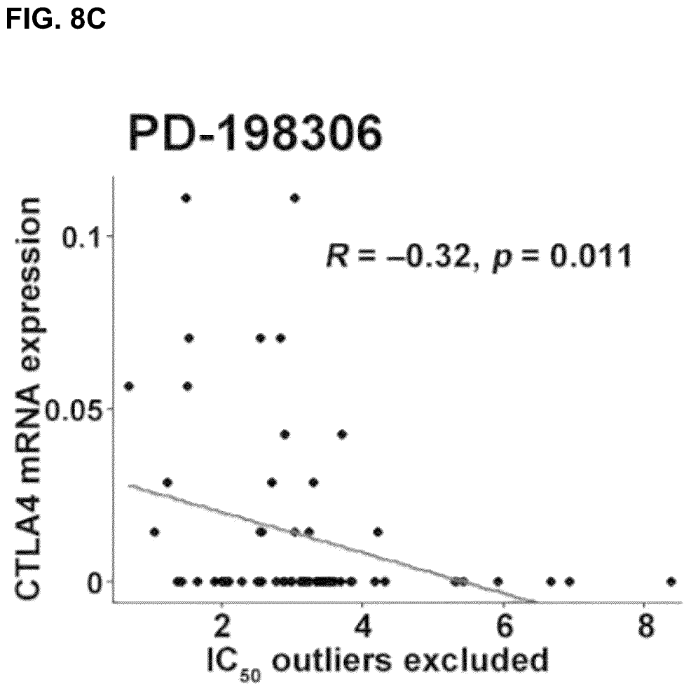

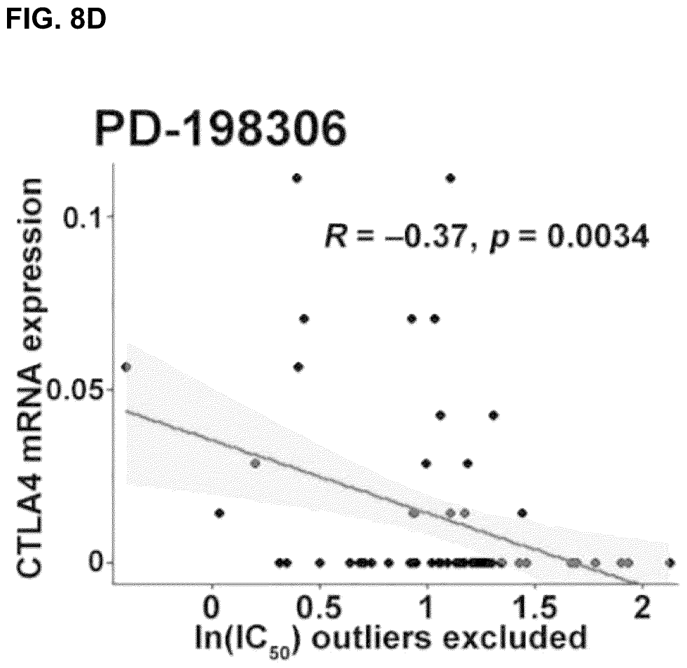

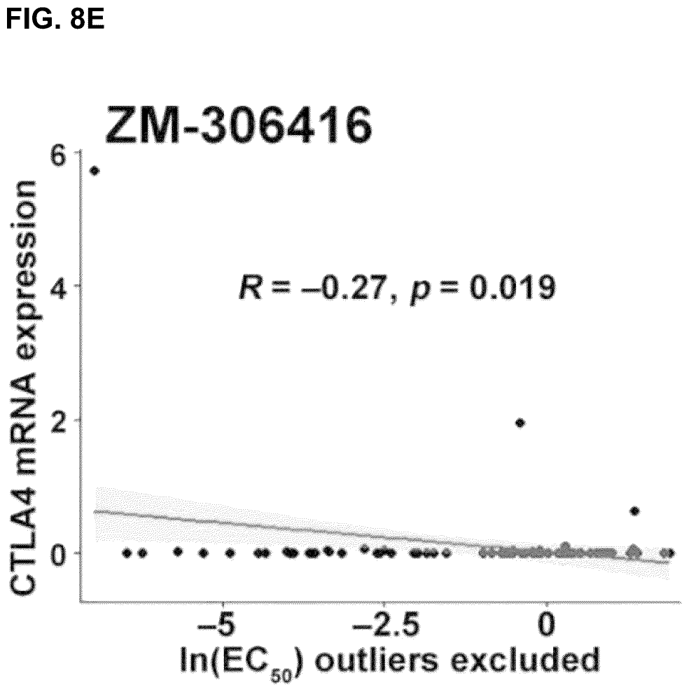

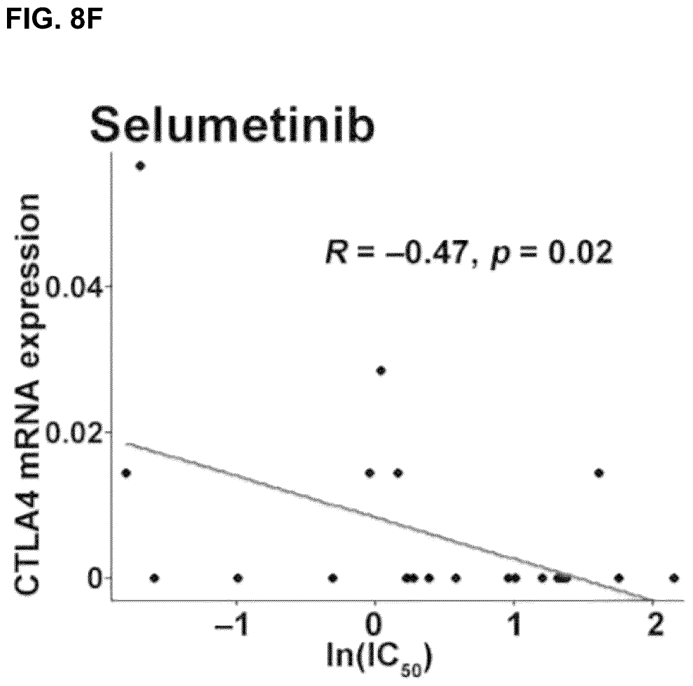

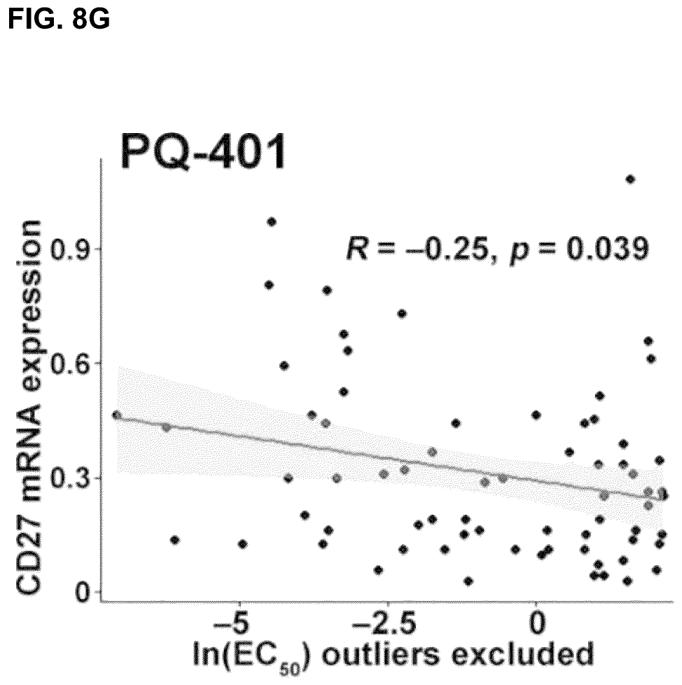

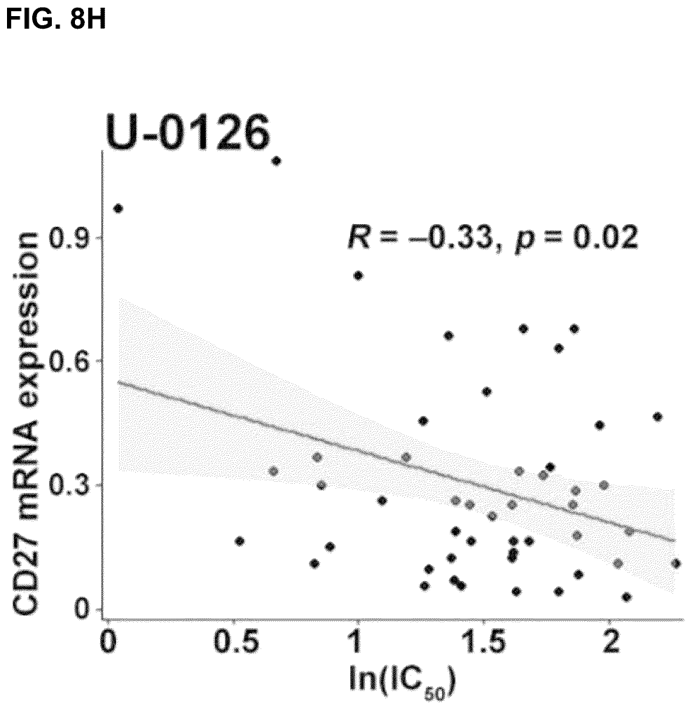

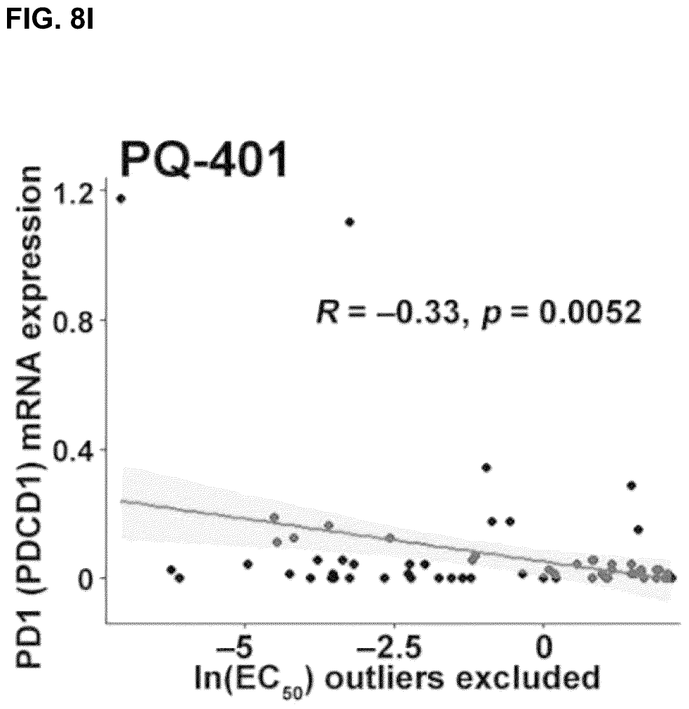

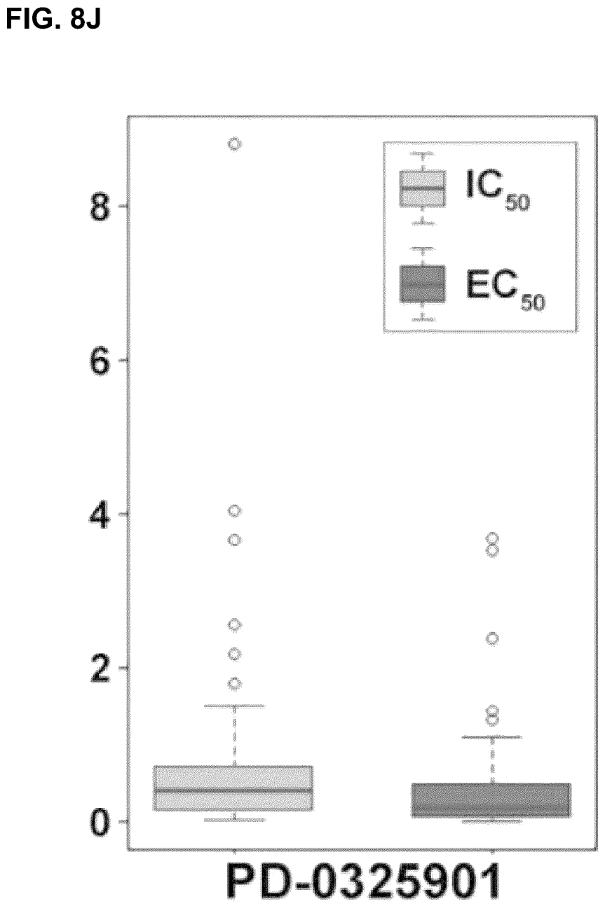

FIG. 8A shows discovering repositioning drugs based on the selected genes. Selection of significant functional pathways and repositioning drugs based on the selected genes.

FIG. 8B shows discovering repositioning drugs based on the selected genes. The Pearson correlations of PDL1 (CD274) protein expression with AS-703026 EC₅₀ excluding outliers.

FIG. 8C shows discovering repositioning drugs based on the selected genes. CTLA4 mRNA expression with PD-198306 IC₅₀ excluding outliers.

FIG. 8D shows discovering repositioning drugs based on the selected genes. PD-198306 ln(IC₅₀) excluding outliers.

FIG. 8E shows discovering repositioning drugs based on the selected genes. ZM-306416 ln(EC₅₀) excluding outliers.

FIG. 8F shows discovering repositioning drugs based on the selected genes. Selumetinib ln(IC₅₀).

FIG. 8G shows discovering repositioning drugs based on the selected genes. CD27 mRNA expression with PQ-401 ln(EC₅₀) excluding outliers.

FIG. 8H shows discovering repositioning drugs based on the selected genes. U-0126 ln(IC₅₀).

FIG. 8I shows discovering repositioning drugs based on the selected genes. PD1 (PDCD1) mRNA expression with PQ-401 ln(EC₅₀) excluding outliers.

FIG. 8J shows discovering repositioning drugs based on the selected genes. Selected compound that had a low average IC₅₀ and EC₅₀ in the CCLE NSCLC cell lines (n=64 [IC₅₀]; n=88 [EC_{50]}).

With the final up- and downregulated genes (FIG. 8A) as CMap input, significantly enriched (p<0.05, connectivity score>0.9) functional pathways (Table 7), compound sets (Table 8), and 28 potential new or repositioning drugs were identified. In addition to PDL1 and CD27, other immune checkpoint inhibitors (ICIs) for treating advanced metastatic NSCLC include anti-PD-1 nivolumab and anti-CTLA4 ipilimumab [39]. To confirm the compound inhibitory effects on ICIs, we further checked if the compounds had a significant negative correlation (p<0.05, R<0, Pearson's correlation test) between drug concentration and mRNA or protein expression of major ICIs for NSCLC treatment, including CD27, CTLA4, PD1 (PDCD1), and PDL1 (CD274) in the CCLE NSCLC cell lines (n=135). EC₅₀ of AS-703026 had a significant negative correlation with PDL1 protein expression in the drug screening data PRISM (FIG. 8B). The drug concentration [IC₅₀, EC₅₀, ln(IC₅₀), or ln(EC₅₀) value] of five compounds, PD-198306, ZM-306416, selumetinib, PQ-401, and U-0126, had a significant negative correlation with mRNA expression of CD27, CTLA4, or PD1, respectively, in the drug screening data PRISM (FIGS. 8C-I).

To investigate if the identified compounds can effectively inhibit the growth of NSCLC cells, the average IC₅₀ and EC₅₀ values in the CCLE NSCLC cell lines (n=135) of the drugs available in PRISM were examined (Table 3). PD-0325901 (FIG. 8J) and dasatinib had small average IC₅₀ and EC₅₀ values in the PRISM drug screening data, implying their potential to inhibit the growth of NSCLC cells with a safe dose.

TABLE 3
Average IC50 and EC50 values of the selected therapeutic compounds in the PRISM dataset. Outliers
(drug concentration value > 10) were removed in the calculation of average IC50 and EC50 values.

| | | | count of | | | count of | |
|---|---|---|---|---|---|---|---|
| | | | IC₅₀ | | | EC₅₀ | |
| | | | (count | | | (count | |
| | | average | of IC₅₀ | | average | of EC₅₀ | |
| src_set_id | compound | of IC₅₀ | outliers) | | of EC₅₀ | outliers) | |
| CP_MEK_INHIBITOR | PD-0325901 | 0.748 | 64 | (0) | 0.386 | 88 | (0) |
| CP_SRC_INHIBITOR | dasatinib | 0.555 | 92 | (0) | 0.504 | 155 | (4) |
| MAP_KINASE_INHIBITOR | PD-98059 | 4.977 | 4 | (0) | 0.607 | 56 | (3) |
| LEUCINE_RICH_REPEAT_KINASE_INHIBITOR | indirubin | 3.548 | 23 | (0) | 0.630 | 160 | (7) |
| CP_MEK_INHIBITOR | selumetinib | 2.214 | 35 | (0) | 0.647 | 165 | (7) |
| CP_MEK_INHIBITOR | AS-703026 | 1.514 | 100 | (2) | 0.940 | 164 | (8) |
| CP_SRC_INHIBITOR | saracatinib | 1.733 | 103 | (0) | 0.983 | 170 | (6) |
| CP_IGF_1_INHIBITOR | BMS-754807 | 1.744 | 97 | (0) | 1.022 | 168 | (8) |
| CP_SRC_INHIBITOR | ZM-306416 | 2.626 | 28 | (0) | 1.081 | 87 | (1) |
| CP_SRC_INHIBITOR | bosutinib | 3.162 | 46 | (0) | 1.587 | 87 | (0) |
| CP_MEK_INHIBITOR | U-0126 | 4.533 | 77 | (0) | 1.868 | 177 | (3) |
| CP_MEK_INHIBITOR | PD-198306 | 3.164 | 67 | (1) | 2.034 | 92 | (0) |
| CP_SRC_INHIBITOR | PP-1 | 3.408 | 51 | (0) | 2.079 | 87 | (0) |
| CP_IGF_1_INHIBITOR | linsitinib | 4.251 | 64 | (1) | 2.088 | 146 | (9) |
| CP_MEK_INHIBITOR | PD-184352 | 3.988 | 47 | (1) | 2.190 | 89 | (0) |
| CP_MEK_INHIBITOR | MEK1-2-inhibitor | 4.197 | 44 | (7) | 2.200 | 84 | (1) |
| IGF-1_INHIBITOR | PQ-401 | 5.344 | 30 | (0) | 2.204 | 79 | (1) |
| CP_SRC_INHIBITOR | PP-2 | 3.187 | 64 | (0) | 2.484 | 79 | (1) |

### 2.8. Potential Oncogenes and Tumor Suppressor Genes

To identify potential oncogenes and tumor suppressor genes among the seven-gene panel, EMT genes, the intracellular innate immune response (IIIR) genes, and all the intermediate genes in previously identified networks (FIGS. 5A-C and FIGS. 7A-D), genes that were differentially expressed between tumors and NATs and were significantly associated with patient survival in LUAD proteomics data (n=103) were selected [33]. Genes that had significantly higher protein expression (p<0.05; two-sample t-tests) in tumors and were survival-hazard (p<0.05, hazard ration [HR]>1; univariate Cox proportional-hazards model) were identified as potential oncogenes. Genes that had significantly higher protein expression (p<0.05; two-sample t-tests) in NATs and were survival-protective (p<0.05, HR<1; univariate Cox proportional-hazards model) were identified as potential tumor suppressor genes. The identified potential oncogenes included EIF4G3, GAPVD1, IKBKB, MCM2, and RFC4. The identified potential tumor suppressor genes included DAG1, SLC39A8, TMEM173/STING1, RDX, TJP1, CTNNB1, and SMC3. Somatic copy number alterations and their correlations with mRNA, protein, and phosphoprotein of the selected genes were extracted from Xu et al [33].

### 3. Discussion

This study further validated the seven-gene panel in patient recurrence risk stratification using proteomic profiles in early-stage NSCLC patients. In addition, we showed that the seven-gene panel can accurately classify tumors from NATs on both RNA sequencing and proteomic platforms, suggesting its potential diagnostic implications for NSCLC. These findings warrant further clinical studies on liquid biopsies in the early detection of NSCLC. Blood-based assays for predicting NSCLC risk and metastasis are important for thoracic surgeons in clinical decision-making, given that the current accepted benign rate is 5% in surgery and 20-30% in biopsies [40]. Developing minimally invasive biomarker-based assays will reduce unnecessary surgeries and biopsies on patients who do not have lung cancer.

In this invention, we further extended our previous findings on prognostic implication of the seven-gene signature for NSCLC patient survival using proteomics/RNA-Seq data of Xu's LUAD (n=103) cohort and RNA-Seq data of TCGA-NSCLC patient cohort (n=923). From the available expression data, we found that ABCC4 was positively associated with patient survival in both Xu's LUAD protein and TCGA-NSCLC RNA-Seq data. DAG1 was positively associated with patient survival at both mRNA and protein levels, while CCL19 and SLC39A8 showed similar associations at the protein level in Xu's LUAD dataset. At the same time, CD27 mRNA expression was associated with worse patient outcomes. Noteworthy, the combined protein expression score of ABCC4, DAG1, and SLC39A8 efficiently stratified patient outcome (p=0.0013, HR: 8.378 [1.774, 39.57]; FIG. 1A), suggesting that these three proteins could potentially be used as prognostic markers in NSCLC. Furthermore, we showed that ABCC4, DAG1, and SLC39A8 proteins are significantly downregulated in tumors compared to NATs (FIGS. 2A-E), suggesting that they may play the role of tumor suppressors.

Expression of ZNF71 was only available at the mRNA level in the TCGA dataset, and we found only a trend for its negative association with patient survival (FIG. 3A), consistent with our previous results in RNA-Seq dataset GSE81089 of NSCLC tumors (n=197) [19]. Based on its structure-inferred function, we hypothesized that ZNF71 could be involved in the suppression of endogenous transposable elements (TEs) expression, which is often activated in cancer and can trigger an innate immune response. We found that overexpression of ZNF71 in A549 lung adenocarcinoma cells resulted in the downregulation of multiple components of the intracellular intrinsic and innate immune systems, including dsRNA (OAS1) and dsDNA (STING, pTBK1) sensors and viral restriction factors (TRIM5, SAMDH1) (FIG. 4). Activation of dsRNA and dsDNA sensors often leads to induction of type I interferons, which can later bridge to adaptive immune response. In particular, type I interferons have been shown to influence the maturation and migration of DC cells, which are important for the cross-priming of NK and CD8+ T cells in the tumor microenvironment [34]. Therefore, we calculated DC xCell score in the TCGA dataset and combined it with ZNF71 expression levels. Interestingly, high expression of ZNF71 and low DC xCell score were associated with worse patient outcomes (FIG. 3B). ZNF71 gene expression is positively associated with immune infiltration including DC and CD8+ T cells in TCGA NSCLC tumors as we previously reportedly [15]. These data indicate a potential interplay between ZNF71 expression in NSCLC and anti-tumor immune response. Thus, future studies should be aimed at the identification of ZNF71 targets and establishing a mechanistic link between ZNF71, innate immune response, and ICI therapy outcomes.

To identify potential new and repositioning medication candidates, we developed mechanisms of action to improve therapy response, extend patient survival, decrease proliferation, and reverse EMT. Therapeutic targets were identified from pan-sensitive genes and pan-resistant genes from the following list: 1) the seven-gene panel, 2) selected epithelial genes and mesenchymal genes, 3) IIIR genes (FIG. 4), and 4) all the genes in the ZNF71-IIIR gene association networks (FIGS. 5-7). These genes are relevant to ZNF71. Direct targets of ZNF71 will be identified from RNA-sequencing of NSCLC cell lines after ZNF71 knockdown/overexpression, which is our ongoing research.

Several MEK1/2 inhibitors were selected as potential targeted therapy also with an inhibitory effect on ICIs for treating NSCLC. Selumetinib is an FDA-approved drug to treat neurofibromatosis type 1 with symptomatic, inoperable plexiform neurofibromas. It is also a designated orphan drug for treating thyroid cancer. Selumetinib is being investigated as a secondary therapy for treating late-stage, metastatic, Kras-mutant NSCLC in several trials [41]. Compared with various combination therapies, chemo-, and immune therapy, selumetinib does not have superior efficacy but does have a better safety profile in treating NSCLC [42]. AS-703026 (pimasertib) has a clinical activity of phosphorylated extracellular signal-regulated kinase (pERK) inhibition in peripheral blood mononuclear cells in patients with locally advanced/metastatic melanoma, particularly BRAF- and NRAS-mutated tumors at clinically relevant doses in a phase I study [43]. In a phase I trial of patients with solid tumors, pimasertib inhibited pERK and the recommended phase II dose (RP2D) was defined as 60 mg bid [44]. PD-198306, an orally active inhibitor of MEK1/2, acts as a potent mitochondrial protonophore and uncouples oxidative phosphorylation [45]. PD-198306 is being studied in rabies virus infection [46], neuropsychiatric disorders [47, 48], breast cancer [49], and osteoarthritis [50] research. Resulting from direct inhibition of MEK1 and MEK2, U-0126 inhibits endogenous promoters containing activator protein 1 (AP-1) response elements but does not affect genes lacking an AP-1 response element in their promoters [51]. U-0126 inhibits anchorage-independent growth of Ki-ras-transformed rat fibroblasts by concurrently suppressing both ERK and mammalian target of rapamycin (mTOR)-p70(S6K) pathways, and sensitizes human breast cancer MDA-MB-231 and HBC-4 cells to anoikis [52].

Among other selected compounds with an in-vitro inhibitory effect on ICIs, ZM-306416 is a VEGFR antagonist and a potent inhibitor of EGFR function [53]. As an inhibitor of placental growth factor (PGF) receptor FLT1, ZM-306416 impaired trophoblast proliferation and migration in fetal growth restriction [54]. PQ-401 is an IGF-IR inhibitor that induces apoptosis and inhibits in-vitro viability, proliferation, and mobility of U87MG glioma cells and in-vivo glioma tumor growth in a mouse xenograft model [55]. In a separate study, PQ-401 inhibits osteosarcoma cell proliferation, migration, and colony formation in U2OS and 143B lines [56]. Overall, the above analysis identified several compounds, including PD-198306, U-0126, ZM-306416, and PQ-401, as potential targeted therapy that may also induce immune response for treating NSCLC, which was not known before.

Dasatinib was reported as a potential repositioning drug for treating NSCLC in our previous publication [15]. PD-0325901 was used to treat refractory NSCLC patients but did not meet its primary endpoint in an open-label, phase II study [57]. Combinations of indirubin and arylidene derivatives showed antimetastatic effects on human NSCLC A549 and NCI-H460 cells [58]. Saracatinib, an orally available inhibitor of src kinases, improved progression-free survival in a subset of patients with advanced, platinum-pretreated NSCLC in a phase II clinical trial [59]. BMS-754807 alone reduced cell survival and wound closure and enhanced apoptosis in human NSCLC A549 and NCI-H358 cells, particularly in NSCLC cells expressing high levels of IGF-IR [60]. In addition, BMS-754807 enhanced cisplatin and carboplatin in A549 cells [60]. A combination of trametinib and bosutinib can synergistically suppress the growth of NSCLC by inhibiting both the mitogen-activated protein kinase (MAPK) and proto-oncogene tyrosine-protein kinase (SRC) pathways, suggesting the potential for treating NSCLC, especially in the treatment of erlotinib-resistant NSCLC [61]. Maintenance therapy of adding linsitinib to erlotinib did not improve PFS or OS in non-progressing NSCLC patients in a phase II randomized trial [62]. In a separate phase II study, adding linsitinib to erlotinib resulted in worse patient outcomes compared with erlotinib alone, suggesting that biomarkers are needed to select responding patients [63]. Morphine, a μ-opioid receptor (MOR) agonist, promoted the growth of NSCLC H460 cells both in-vitro and in-vivo, a higher morphine dosage shortens the survival time of patients with lung cancer [64]. Treatment with the Src inhibitor protein phosphatase 1 (PP-1) and the MOR antagonist methylnaltrexone (MNTX) decreased the phosphorylation induced by morphine. Furthermore, the antiapoptotic impact of morphine on NSCLC cells was reversed by MNTX, PP-1, and the PI3K/AKT inhibitor deguelin. Lapatinib (EGFR and HER2 tyrosine kinase inhibitor (TKI)), gefitinib (EGFR TKI), ZD4054 or BQ-123 (ETAR antagonist), GM6001 (matrix metalloprotease inhibitor), PP-2 (Src inhibitor) or Tiron (superoxide scavenger) all inhibited the increase in EGFR and HER2 transactivation induced by the addition of ET-1 to NSCLC cells [65]. These results indicate that our AI pipeline is capable to select relevant compounds for further clinical studies.

Among the identified potential NSCLC oncogenes, a reduction in the germline copy number of EIF4G3 is linked to breast cancer susceptibility in the Japanese population [66]. IkappaB kinase (IKK) promotes tumorigenesis via inhibiting forkhead FOXO3a which can be reversed by FOXO3a [67]. Conditional suppression of IKBKB inhibits melanoma tumor development in mice, and IKBKB-mediated NFkB activity is required in mutant Hras-initiated tumorigenesis [68]. IKBKB also promotes osteosarcoma cancer progression [69]. Minichromosome maintenance proteins (MCMs) are essential in DNA replication, genomic stability, and cell proliferation [70, 71]. MCMs, in particular, MCM2 and MCM4, are potential biomarkers to identify high-risk NSCLC patients [72]. MCM2 was significantly overexpressed in almost all human cancers/subtypes in TCGA and was associated with tumor mutation burden, tumor stage, immune therapy response, immune infiltration, and patient poor prognosis [73]. RFC4 is frequently overexpressed in colorectal cancer (CRC), and RFC4 overexpression is associated with tumor progression and shorter patient survival, possibly due to RFC4-mediated cell cycle arrest and the regulation of CRC cell proliferation [74]. RFC4, along with other genes and microRNAs, might promote osteosarcoma initiation and development [75]. GAPVD1, a cytoplasmic trafficking factor, is involved in the regulation of the mammalian circadian clock [76]. Mutations in GAPVD1 and other genes, estrogen- and growth factor-dependent regulation are involved in both transcriptional and post-transcriptional dysregulation of syndecan-4 in breast cancer [77]. Together, our identified NSCLC oncogene genes are supported by the literature.

Among the identified potential tumor suppressor genes, DAG1 was co-deleted with the Von Hippel Lindau (VHL) tumor suppressor gene in clear cell renal cell carcinoma [78]. In glioblastomas, DAG1 correlated with tumor grade, and the patient group with higher expression of DAG1 survived shorter than the patient group with lower expression of DAG1 [79]. These results suggest that DAG1 may have different functions in tumor initiation and progression in different cancer types. SLC39A8 is responsible for pasting zinc to the cytoplasm when zinc is depleted for maintaining many critical biological processes. SLC39A8 suppresses the progression of clear cell renal cell carcinoma [80]. TMEM173/STING1 was expressed higher in normal samples in lung adenocarcinoma, lung squamous carcinoma, prostate adenocarcinoma, uterine corpus endometrial carcinoma, but was expressed higher in tumor tissues in colorectal carcinoma, kidney renal clear cell carcinoma, stomach adenocarcinoma, and thyroid adenocarcinoma [81]. MTA1-induced inhibition of TJP1 protein co-localized in the cytoplasm and membrane of NSCLC cells leads to weakened cell junctions and changes in adhesion, migration, and invasion capabilities of cells, putatively promoting the invasion and metastasis of NSCLC [82]. β-catenin/CTNNB1 is an intracellular scaffold protein. Aberrant CTNNB1 signaling is one of the fundamental processes in many human cancers [83, 84]. Both gain-of-function and loss-of-function CTNNB1 mutations are found in multiple human cancer types [85]. SMC3 haploinsufficiency accelerates lymphomagenesis in mice with constitutive BCL6 expression and is considered a putative tumor suppressor for germinal center B cells [86]. RDX knockdown increased the intracellular SN-38 concentration, indicating enhanced anti-tumor activity, in human clear cell renal cell carcinoma Caki-1 cells [87]. To date, the literature supports the tumor suppressor functions of DAG1, SLC39A8, TMEM173/STING1, TJP1, CTNNB1, and SMC3, but not RDX.

### 4. Materials and Methods

### 4.1. Non-Small Cell Lung Cancer (NSCLC) Patient Cohorts

This study obtained NSCLC patient sample data from public resources including Xu et al [33] and The Cancer Genome Atlas (TCGA Research Network: https://www.cancer.gov/tcga, accessed on 28 Apr. 2021). The primary lung adenocarcinoma (LUAD) cohort collected samples from 103 randomly selected treatment-naïve Chinese patients between 2010 and 2016 from Xu et al [33]. Proteomics data of 103 paired LUAD tumors and non-cancerous adjacent tissues (NATs), and RNA sequencing data of 51 paired LUAD tumors and 49 NATs were used in this study. RNA sequencing data of TCGA NSCLC patient cohort, i.e. TCGA-LUAD (n=515) and TCGA-LUSC (n=501), with patient clinical information were downloaded from an openly accessible entry LinkedOmics (http://www.linkedomics.org, accessed on 28 Apr. 2021) [88].

4.2. xCell

The xCell (https://xcell.ucsf.edu/, accessed on 12 Jul. 2022) tool [89] was used to predict the levels for 64 immune and stroma cell types based on gene expression data. The xCell scores for patient samples were calculated using single-sample gene set enrichment analysis (ssGSEA) to analyze the immune microenvironment. Low xCell scores indicated the cell type had similar levels across all samples; whereas high xCell scores indicated the cell type had different levels across all samples

### 4.3. Weka

The Weka software (Version 3.8.6) [90] was utilized to conduct machine learning classifier approaches to differentiate between tumors and NATs with selected genes in Xu's LUAD RNA sequencing and proteomics data [33]. Commonly used machine learning classification methods, including decision tree, k-nearest neighbors (KNN), logistic regression, naïve Bayes, random forests, support vector machine (SVM), and radial basis function (RBF) network, were used. Ten-fold cross-validation was applied in each session.

### 4.4. Cell Lines

A549 cells (kind gift of Dr. Ivan Martinez, WVU) were grown in DMEM (Corning, cat. #15-018-CV) supplemented with 10% FBS (HyClone, UT, USA), 2 mM L-glutamine (Corning, cat. #25-005-CI), and 1×Antibiotic Antimycotic Solution (Corning, cat. #30-004-CI). All cells were maintained at 37 C in a 5% CO2 incubator.

### 4.5. Vector Construction and Lentiviral Transduction

ZNF71 KRABless isoform was PCR-amplified from cDNA obtained from MDA231 cells using PfuUltraII DNA polymerase using primers #5ZNF71 ex1-Mun: 5′-AGAGCAATT-GATGGCTGCTCAGCTGC-3′ and #3ZNF71-X: 5′-AGACTCGAGTCAGGTGTGAATCCG-CAG-3′ and cloned into Dox-inducible pLUT lentiviral vector using EcoRI and XhoI cloning sites. To generate the KRAB isoform, a 397 bp long KRAB containing 5′-terminal fragment was synthesized at GenScript and cloned into pLUT-ZNF71-KRABless using NheI and EcoRI sites. The cloned cDNAs were sequenced to verify the absence of mutations.

Lentiviral particles were packaged in HEK293T cells (RRID:CVCL_0063) following calcium phosphate cotransfection of constructed pLUT-ZNF71 vectors, psPAX2 (Addgene, 12260), and pCMV-VSV-G (Addgene, 8454) as previously described [91]. pLUT vector expressing turbo red fluorescent protein (RFP) was used as control. After two rounds of infection, transduced A549 cells were selected in puromycin (1 ug/mL) containing media for at least 5 days Ectopic ZNF71 expression was induced by culturing cells in media containing 0.5 ug/mL doxycycline for 7 days.

### 4.6. Western Blot

Whole-cell lysates were prepared in nonreducing Laemmli buffer as described in [91]. Protein concentration was quantified by Pierce BCA Protein Assay (ThermoFisher, cat #23225). Lysates with an equal amount of total protein were separated on 4% to 12% Bis-Tris NuPAGE Novex gels and transferred to a polyvinylidene difluoride (PVDF) membrane. Protein bands were detected using standard chemiluminescence techniques using GE Healthcare Amersham Imager 680.

The following antibodies were used in Western blotting: ZNF71 (GeneTex, cat. #GTX116553), from Cell Signaling Biotechnology: STING (cat. #13647), TBK1 pSer172 (cat. #5483), TBK1 (cat. #3504), OAS1 (cat. #14498), RNase L (cat. #27281), TRIM5α (cat. #14326), SAMHD1 pThr592 (cat. #89930), and GAPDH (Millipore, MAB374).

### 4.7. Boolean Implication Network

The Boolean implication network algorithm [92-94] was used in this study to generate gene associate networks. Details of the algorithm were included in our previous publications [15, 95]. Thresholds of scope and precision for filtering the implication rules were computed based on a one-tailed z-score of 1.64 (95% level of significance).

Xu's LUAD cohort [33] was used to construct the gene association networks. Each gene's expression was divided into three categories: under-expressed (−1), normal (0), and over-expressed (1). The categorization was based on the distribution of the selected housekeeping genes (B2M, ESD, FLOT2, GAPDH, GRB2, HPRT1, HSP90AB1, LDHA, NONO, POLR2A, PPP1CA, RHOA, SDCBP, and TFRC) [14, 96-98]. The percentage of under-expressed and over-expressed samples for all the housekeeping genes was fixed to be 30% in each dataset. Standard deviations were calculated for the normal range based on the housekeeping genes and applied to the rest genes. The numbers of standard deviation used for each dataset were: 0.68 for RNA sequencing data of LUAD tumors; 0.75 for RNA sequencing data of NATs; 0.88 for proteomics and RNA sequencing data of LUAD tumors; 0.95 for proteomics data and RNA sequencing data of NATs. The networks were visualized with Cytoscape (version 3.9.1) [99].

### 4.8. CRISPR-Cas9 Knockout Assays

Genome-scale CRISPR-Cas9 knockdown data from project Achilles [100, 101] were obtained from the DepMap (release 21Q4) [102]. CRISPR-Cas9 dependency scores of human NSCLC cell lines (n=94) from the DepMap portal (https://depmap.org/portal/download/all/, accessed on 12 Sep. 2022) were used in this study. The CERES method was used to normalize gene-level dependency scores. The median of the normalized dependency scores of common essential genes was −1.0, and that of non-essential genes was 0 in each cell line. In this study, a dependency score less than −0.5 indicated that the gene had a significant effect on the cell line in CRISPR-Cas9 knockout.

### 4.9. RNAi Knockdown Assays

Genome-wide RNA interference (RNAi) knockdown screening data [103] in project Achilles was analyzed in this study. RNAi dependency scores were processed with the DEMETER2 v6 algorithm [103] to distinguish between on- and off-target effects. The dependency scores of human NSCLC cell lines (n=92) were obtained from the DepMap portal (https://depmap.org/portal/download/all/, accessed on 12 Sep. 2022). The median of the normalized dependency scores of the positive control gene set was −1, and that of the negative control gene set was 0. The gene that had a dependency score less than −0.5 was considered as having a significant effect on the cell line in RNAi knockdown.

### 4.10. Pathway Enrichment Analysis Using ToppGene

The ToppFun web tool (https://toppgene.cchmc.org/enrichment.jsp, accessed on 28 Jun. 2022) from the ToppGene suite [104] is an online resource for gene list enrichment analysis. We used the ToppGene application with default parameters (FDR correction, p-value cutoff=0.05, gene limits 1≤n≤2000) to perform pathway enrichment analysis.

### 4.11. Cancer Cell Line Encyclopedia (CCLE)

RNA sequencing data of 135 human NSCLC cell lines were obtained from the Cancer Cell Line Encyclopedia (CCLE) in DepMap release 22Q2 (https://depmap.org/portal/download/all/, accessed on 12 Sep. 2022). Proteomics data of 64 human NSCLC cell lines were obtained from Nusinow et al [105].

### 4.12. Drug Sensitivity in CCLE

The drug sensitivity data of human NSCLC cell lines were taken from multiple resources. The secondary PRISM repurposing dataset [35] was obtained from the DepMap release 19Q4. It has 1,448 compounds screened in 499 human cell lines, 84 of which were NSCLC cell lines and were used in this study. The Genomics of Drug Sensitivity in Cancer (GDSC) datasets (GDSC1 and GDSC2 [36-38]) were downloaded (https://www.cancerrxgene.org/downloads/bulk_download, accessed on 12 Sep. 2022). A total of 64 NSCLC cell lines from GDSC1 and 58 NSCLC cell lines from GDSC2 were included in this study. Details of the categorization of drug sensitivity in each cell line were included in our previous publications [15, 95].

### 4.13. Drug Repurposing Using Connectivity Map (CMap)

The connectivity map (CMap) online tool (https://clue.io/, accessed on 12 Sep. 2022) [31, 32] was used to explore functional pathways and potential repositioning of drugs with selected gene expression signatures. Raw connectivity scores higher than 0.9 and a p-value<0.05 were considered significant.

### 4.14. Statistical Methods

Statistical analysis was performed using R software (version 4.1.3) with RStudio (version 2022.02.3 Build 492) [106]. Two sample t-tests (two-tailed) were performed for the comparisons of two groups of continuous variables. The independence of categorical variables was evaluated with χ² tests. Principle component analysis (PCA) was used to generate the separation of NATs and LUAD tumors with selected genes. Visualization was carried out in R and Cytoscape. Kaplan-Meier method was performed to conduct survival analysis and create survival curves. The difference between the survival rates of patient groups was evaluated using log-rank tests. Univariate and multivariate Cox regression analyses were performed to evaluate the prognostic capacity of the studied factors. R packages survival, survminer, and ggplot2 were used in survival analysis. Pearson's correlation coefficients were used to determine the association between the two sample groups. All hypothesis tests were two-sided, and test results with a p-value<0.05 were considered statistically significant.

### 5. Conclusions

This invention extended a seven-gene panel for NSCLC prognosis using proteomic profiles. The results also showed that the seven-gene panel can accurately classify NSCLC tumors from NATs on both RNA-sequencing and proteomic platforms, suggesting its diagnostic implications for the early detection of lung cancer. Gene expression of ZNF71, a marker within the seven-gene panel, when combined with dendritic cell activities, can further stratify NSCLC into different prognostic groups. ZNF71 expression is associated with the activities of NK cells and NKT cells. Overexpression of ZNF71 results in decreased expression of multiple components of the intracellular intrinsic and innate immune systems, including dsRNA and dsDNA sensors, confirming a hypothesis that ZNF71 suppresses the transcription of genomic transposable elements. Multi-omics networks of ZNF71 and the intracellular innate immune response genes were revealed in NSCLC using a computational Boolean implication network algorithm. From these constructed networks, pan-sensitive and pan-resistant genes to 21 NCCN-recommended drugs for treating NSCLC were selected. We designed mechanisms of action to enhance treatment response, prolong patient survival, inhibit proliferation, and reverse EMT to screen candidates for new and repositioning drugs. PD-198306, U-0126, ZM-306416, and PQ-401 were identified as potential targeted therapy that may also induce immune response for treating NSCLC, which was not known before. Our future research will identify direct targets of ZNF71 for the development of novel therapeutic strategies to improve NSCLC survival outcomes.

TABLE 4
Machine learning classification of tumors vs. NATs in RNA sequencing data of
lung adenocarcinoma patients (n = 51) from Xu et al. Genes used in these
classification methods included ABCC4, CCL19, CD27, DAG1, FUT7, and ZNF71.
Classification methods were performed with Weka in 10-fold cross-validation.

| | Decision | | Logistic | Naïve | Random | | RBF |
|---|---|---|---|---|---|---|---|
| | tree | KNN | regression | Bayes | forests | SVM | Network |
| Sensitivity | 0.765 | 0.824 | 0.824 | 0.745 | 0.882 | 0.961 | 0.824 |
| Specificity | 0.714 | 0.755 | 0.837 | 0.939 | 0.837 | 0.490 | 0.898 |
| PPV | 0.736 | 0.778 | 0.840 | 0.927 | 0.849 | 0.662 | 0.894 |
| NPV | 0.745 | 0.804 | 0.820 | 0.780 | 0.872 | 0.923 | 0.830 |
| Overall | 0.740 | 0.790 | 0.830 | 0.840 | 0.860 | 0.730 | 0.860 |
| Accuracy | | | | | | | |
| ROC Area | 0.759 | 0.789 | 0.915 | 0.920 | 0.927 | 0.725 | 0.896 |
| Odds Ratio | 8.13 | 14.39 | 23.92 | 44.82 | 38.44 | 23.52 | 41.07 |

TABLE 5
Machine learning classification of tumors vs. NATs in proteomics data
of lung adenocarcinoma patients (n = 103) from Xu et al. Genes used
in these classification methods included ABCC4, DAG1, and SLC39A8. Classification
methods were performed with Weka in 10-fold cross-validation.

| | Decision | | Logistic | Naïve | Random | | RBF |
|---|---|---|---|---|---|---|---|
| | tree | KNN | regression | Bayes | forest | SVM | Network |
| Sensitivity | 0.800 | 0.822 | 0.867 | 0.911 | 0.822 | 0.867 | 0.889 |
| Specificity | 0.967 | 0.944 | 0.956 | 0.922 | 0.922 | 0.978 | 0.956 |
| PPV | 0.923 | 0.881 | 0.907 | 0.854 | 0.841 | 0.951 | 0.909 |
| NPV | 0.906 | 0.914 | 0.935 | 0.954 | 0.912 | 0.936 | 0.945 |
| Overall | 0.911 | 0.904 | 0.926 | 0.919 | 0.889 | 0.941 | 0.933 |
| Accuracy | | | | | | | |
| ROC Area | 0.897 | 0.879 | 0.957 | 0.967 | 0.959 | 0.922 | 0.949 |
| Odds Ratio | 116 | 78.63 | 139.75 | 121.54 | 54.84 | 286 | 172 |

TABLE 6
Proliferation and differential expression status of the seven-
gene panel and intracellular immune response (IIIR) genes.

| | | Percentage of | Percentage of | | |
|---|---|---|---|---|---|
| | | significant (<−0.5) | significant (<−0.5) | Xu's LUAD | |
| | | dependency score in | dependency score in | protein log10 | Xu's LUAD |
| Type | Gene | RNAi screening | CRISPR-Cas9 screening | transformed | RNA sequencing |
| 7-gene | ABCC4 | 0.00% | 0.00% | Higher in normal | Higher in tumor |
| 7-gene | CCL19 | 0.00% | 0.00% | Higher in tumor | Higher in tumor |
| 7-gene | CD27 | 0.00% | 0.00% | Not significant | Higher in tumor |
| 7-gene | DAG1 | 0.00% | 0.00% | Higher in normal | Not significant |
| 7-gene | FUT7 | 0.00% | 0.00% | NA | Higher in normal |
| 7-gene | SLC39A8 | 0.00% | 0.00% | Higher in normal | NA |
| 7-gene | ZNF71 | 0.00% | 0.00% | NA | Not significant |
| IIR gene | AIM2 | 3.26% | 0.00% | Not significant | NA |
| IIR gene | CDK1 | 98.91% | 100.00% | Not significant | Higher in tumor |
| IIR gene | EIF2A | 0.00% | 0.00% | Higher in tumor | Higher in tumor |
| IIR gene | EIF2AK2 | 0.00% | 0.00% | Higher in tumor | NA |
| IIR gene | IFNA16 | 0.00% | 0.00% | NA | NA |
| IIR gene | IFNA17 | 0.00% | NA | NA | NA |
| IIR gene | IFNA21 | 0.00% | NA | NA | NA |
| IIR gene | IFNA22P | 0.00% | 0.00% | NA | NA |
| IIR gene | IFNA4 | 0.00% | 0.00% | NA | NA |
| IIR gene | IFNA5 | 15.22% | 0.00% | NA | NA |
| IIR gene | IFNAR1 | 0.00% | 0.00% | Not significant | Higher in normal |
| IIR gene | IFNAR2 | 0.00% | 0.00% | Not significant | Higher in tumor |
| IIR gene | IFNE | 5.43% | 0.00% | NA | Not significant |
| IIR gene | IFNG | 0.00% | 0.00% | NA | NA |
| IIR gene | IFNG-AS1 | 0.00% | 0.00% | NA | Higher in tumor |
| IIR gene | IFNGR1 | 0.00% | 0.00% | Higher in normal | Higher in normal |
| IIR gene | IFNGR2 | 0.00% | 0.00% | NA | Higher in tumor |
| IIR gene | IFNK | 0.00% | 0.00% | NA | NA |
| IIR gene | IFNL1 | 0.00% | 0.00% | NA | NA |
| IIR gene | IFNL3 | 0.00% | 0.00% | NA | NA |
| IIR gene | IFNLR1 | 0.00% | 0.00% | NA | Not significant |
| IIR gene | IFNW1 | 1.09% | 0.00% | NA | NA |
| IIR gene | IKBKB | 0.00% | 0.00% | Higher in tumor | Higher in tumor |
| IIR gene | IRF3 | 1.09% | 0.00% | Higher in tumor | Higher in normal |
| IIR gene | IRF7 | 2.17% | 0.00% | Not significant | Higher in tumor |
| IIR gene | JUN | 21.74% | 12.77% | Not significant | Higher in normal |
| IIR gene | MAP3K7 | 0.00% | 5.32% | Higher in tumor | Higher in tumor |
| IIR gene | MB21D1 | 1.37% | NA | Higher in tumor | NA |
| IIR gene | OAS1 | 0.00% | 0.00% | Not significant | Higher in tumor |
| IIR gene | PFKL | 0.00% | 0.00% | Higher in tumor | NA |
| IIR gene | RNASEL | 1.09% | 0.00% | Higher in tumor | Higher in tumor |
| IIR gene | SAMHD1 | 1.37% | 3.19% | Higher in tumor | Higher in normal |
| IIR gene | TBK1 | 1.09% | 0.00% | Higher in tumor | Higher in tumor |
| IIR gene | TMEM173 | 8.22% | NA | Higher in normal | NA |
| IIR gene | TRIM28 | 13.04% | 1.06% | Higher in tumor | NA |
| IIR gene | TRIM5 | 0.00% | 0.00% | Not significant | NA |

NA—Not available in the data.

TABLE 7
Significantly (p < 0.05, connectivity score > 0.9) enriched functional pathways in analysis with CMap.

| src_set_id | cell_iname | pert_type | genes |
|---|---|---|---|
| BIOCARTA_AKAPCENTROSOME_{—} | HCC515 | TRT_SH.CGS | AKAP9, CDK1, MAP2, NUP85, PCNT, PKN1, |
| PATHWAY | | | PPP2CA, PRKACB, PRKACG, PRKAG1, |
| | | | PRKAR2A, PRKAR2B, PRKCE, RHOA |
| BIOCARTA_BARR_MAPK_{—} | A549 | TRT_SH.CGS | ADCY1, ARRB1, DNM1, GNAS, GRK2, KCNA1, |
| PATHWAY | | | KCNA2, KCNA3, MAP2K1, MAP2K2, MAPK1, |
| | | | MAPK3, PLCB1, RAF1 |
| BIOCARTA_RB_PATHWAY | A549 | TRT_SH.CGS | ATM, CDC25A, CDC25B, CDC25C, CDK1, CDK2, |
| | | | CDK4, CHEK1, MYT1, RB1, TP53, WEE1, |
| | | | YWHAH |
| KD_CYCLIN_DEPENDENT_{—} | A549 | TRT_SH.CGS | CDK2, CDK4, CDK6, CDK9, CDKL4, CDK10 |
| KINASES | | | |
| KD_CYCLINS | A549 | TRT_SH.CGS | CCNL1, CCND1, CCNA1, CCNH |
| KD_INTEGRIN_SUBUNITS_{—} | A549 | TRT_XPR | ITGB1, ITGB4, ITGB5 |
| BETA | | | |
| KD_PHOSPHOLIPASES | A549 | TRT_SH.CGS | PLCB1, PLA2G2A, PLCG1, PLD2 |
| OE_NADH_UBIQUINONE_{—} | A549 | TRT_SH.CGS | NDUFS3, NDUFS7, NDUFV2 |
| OXIDOREDUCTASE_CORE_{—} | | | |
| SUBUNITS | | | |
| OE_PHOSPHOLIPASES | A549 | TRT_SH.CGS | PLCG2, PLA2G12B, PLCB1, PLD1 |
| PID_VEGF_VEGFR_PATHWAY | HCC515 | TRT_SH.CGS | FLT1, FLT4, KDR, NRP1, NRP2, PGF, VEGFA, |
| | | | VEGFB, VEGFC, VEGFD |
| REACTOME_GAP_JUNCTION_{—} | A549 | TRT_SH.CGS | ACTB, ACTG1, AP2M1, CLTA, CLTB, CLTC, |
| DEGRADATION | | | CLTCL1, DAB2, DNM1, DNM2, GJA1, MYO6 |
| REACTOME_HYALURONAN_{—} | A549 | TRT_SH.CGS | CD44, CHP1, GUSB, HEXA, HEXB, HMMR, |
| UPTAKE_AND_DEGRADATION | | | HYAL1, HYAL2, HYAL3, LYVE1, SLC9A1, STAB2 |
| REACTOME_TRAF3_DEPENDENT_{—} | A549 | TRT_SH.CGS | CREBBP, DDX58, EP300, IFIH1, IFNB1, IKBKE, |
| IRF_ACTIVATION_{—} | | | IRF3, IRF7, MAVS, RNF135, SIKE1, TBK1, |
| PATHWAY | | | TRAF3, TRIM25, TRIM4 |

TABLE 8
Significantly (p < 0.05, connectivity score > 0.9) enriched compound sets in the analysis with CMap.

| src_set_id | cell_iname | pert_type | compounds |
|---|---|---|---|
| CP_IGF_1_INHIBITOR | A549 | TRT_CP | BMS-536924, BMS-754807, linsitinib |
| CP_MEK_INHIBITOR | HCC515 | TRT_CP | MEK1-2-inhibitor, PD-198306, PD-98059, U-0126, |
| | | | selumetinib, PD-184352, PD-0325901, AS-703026 |
| CP_SRC_INHIBITOR | HCC515 | TRT_CP | PP-1, PP-2, ZM-306416, dasatinib, saracatinib, WH- |
| | | | 4023, bosutinib |
| IGF-1_INHIBITOR | A549 | TRT_CP | I-OMe-AG-538, tyrphostin-AG-538, PQ-401, BMS- |
| | | | 536924, BMS-754807, linsitinib, GSK-1904529A, EI-247 |
| LEUCINE_RICH_REPEAT_{—} | A549 | TRT_CP | GW-5074, indirubin, XMD-1150, XMD-885 |
| KINASE_INHIBITOR | | | |
| MAP_KINASE_INHIBITOR | HCC515 | TRT_CP | CGP-57380, PD-198306, PD-98059, rottlerin, PD- |
| | | | 0325901, XMD-892, XMD-885 |
| PPAR_RECEPTOR_ANTAGONIST | HCC515 | TRT_CP | bisphenol-a, mifobate, GW-6471, GW-9662, T-0070907, |
| | | | GSK-0660 |
| SULFONYLUREA | HCC515 | TRT_CP | glipizide, glibenclamide, gliquidone |
| VASOPRESSIN_RECEPTOR_{—} | A549 | TRT_CP | relcovaptan |
| ANTAGONIST | | | |

A method of diagnosis and screening of non-small cell lung cancer is provided comprising using protein expression levels of one elected from the group consisting of ABCC4, DAG1, and SLC39A8 in patient samples, of ABCC4 and DAG1 in patient samples, of ABCC4 and SLC39A8 in patient samples, of DAG1 and SLC39A8 in patient samples, of ABCC4 in patient samples, of DAG1 in patient samples, and of SLC39A8 in patient samples.

A method of diagnosis and screening of non-small cell lung cancer is provided comprising using mRNA expression levels of ABCC4, CCL19, CD27, DAG1, FUT7, and ZNF71 in patient samples.

### REFERENCES

1. Lung Cancer-Non-Small Cell: Statistics. May 23, 2022]; Available from: https://www.cancer.net/cancer-types/lung-cancer-non-small-cell/statistics.
2. Ho, C., et al., Histologic classification of non-small-cell lung cancer over time: reducing the rates of not-otherwise-specified. Curr Oncol, 2015. 22(3): p. e164-70.
3. Zappa, C. and S. A. Mousa, Non-small cell lung cancer: current treatment and future advances. Transl Lung Cancer Res, 2016. 5(3): p. 288-300.
4. NCCN Guidelines, version: 3.2022. Jun. 28, 2022]; Available from: https://www.nccn.org/guidelines/category_1.
5. Zheng, Y., M. T. Jaklitsch, and R. Bueno, Neoadjuvant Therapy in Non-Small Cell Lung Cancer. Surg Oncol Clin N Am, 2016. 25(3): p. 567-84.
6. Patsoukis, N., et al., Revisiting the PD-1 pathway. Sci Adv, 2020. 6(38).
7. Chaft, J. E., et al., Evolution of systemic therapy for stages I-III non-metastatic non-small-cell lung cancer. Nat Rev Clin Oncol, 2021. 18(9): p. 547-557.
8. FDA approves atezolizumab as adjuvant treatment for non-small cell lung cancer. 2021 Oct. 18, 2021]; Available from: https://www.fda.gov/drugs/resources-information-approved-drugs/fda-approves-atezolizumab-adjuvant-treatment-non-small-cell-lung-cancer.
9. Hoffman, P. C., A. M. Mauer, and E. E. Vokes, Lung cancer. Lancet, 2000. 355(9202): p. 479-485.
10. Naruke, T., et al., Prognosis and survival in resected lung carcinoma based on the new international staging system. J. Thorac. Cardiovasc. Surg, 1988. 96(3): p. 440-447.
11. Vogt, U., et al., Lack of squamous cell lung carcinoma in vitro chemosensitivity to various drug regimens in the adenosine triphosphate cell viability chemosensitivity assay. Acta Biochim. Pol, 1999. 46(2): p. 299-302.
12. Byron, E. and M. Pinder-Schenck, Systemic and targeted therapies for early-stage lung cancer. Cancer Control, 2014. 21(1): p. 21-31.
13. Rena, O., et al., Stage I non-small cell lung carcinoma: really an early stage? Eur J Cardiothorac Surg, 2002. 21(3): p. 514-9.
14. Guo, N. L., et al., A Predictive 7-Gene Assay and Prognostic Protein Biomarkers for Non-small Cell Lung Cancer. EBioMedicine, 2018. 32: p. 102-110.
15. Ye, Q., et al., A Multi-Omics Network of a Seven-Gene Prognostic Signature for Non-Small Cell Lung Cancer. Int J Mol Sci, 2021. 23(1).
16. Waldman, A. D., J. M. Fritz, and M. J. Lenardo, A guide to cancer immunotherapy: from T cell basic science to clinical practice. Nature Reviews Immunology, 2020. 20(11): p. 651-668.
17. Marin-Acevedo, J. A., et al., Next generation of immune checkpoint therapy in cancer: new developments and challenges. J Hematol Oncol, 2018. 11(1): p. 39.
18. Burris, H. A., et al., Safety and Activity of Varlilumab, a Novel and First-in-Class Agonist Anti-CD27 Antibody, in Patients With Advanced Solid Tumors. J Clin Oncol, 2017. 35(18): p. 2028-2036.
19. Ye, Q., et al., Molecular Analysis of ZNF71 KRAB in Non-Small-Cell Lung Cancer. Int J Mol Sci, 2021. 22(7).
20. Bruno, M., M. Mahgoub, and T. S. Macfarlan, The Arms Race Between KRAB-Zinc Finger Proteins and Endogenous Retroelements and Its Impact on Mammals. Annu Rev Genet, 2019. 53: p. 393-416.
21. Sadeq, S., et al., Endogenous Double-Stranded RNA. Noncoding RNA, 2021. 7(1).
22. Kassiotis, G., Endogenous retroviruses and the development of cancer. J Immunol, 2014. 192(4): p. 1343-9.
23. Chiappinelli, K. B., et al., Inhibiting DNA Methylation Causes an Interferon Response in Cancer via dsRNA Including Endogenous Retroviruses. Cell, 2015. 162(5): p. 974-86.
24. Di Giorgio, E. and L. E. Xodo, Endogenous Retroviruses (ERVs): Does RLR (RIG-I-Like Receptors)-MAVS Pathway Directly Control Senescence and Aging as a Consequence of ERV De-Repression? Front Immunol, 2022. 13: p. 917998.
25. Hur, S., Double-Stranded RNA Sensors and Modulators in Innate Immunity. Annu Rev Immunol, 2019. 37: p. 349-375.
26. Yang, E. and M. M. H. Li, All About the RNA: Interferon-Stimulated Genes That Interfere With Viral RNA Processes. Front Immunol, 2020. 11: p. 605024.
27. Kim, Y., et al., PKR Senses Nuclear and Mitochondrial Signals by Interacting with Endogenous Double-Stranded RNAs. Mol Cell, 2018. 71(6): p. 1051-1063.e6.
28. Chamontin, C., et al., Regulation of Viral Restriction by Post-Translational Modifications. Viruses, 2021. 13(11).
29. Harding, S. M., et al., Mitotic progression following DNA damage enables pattern recognition within micronuclei. Nature, 2017. 548(7668): p. 466-470.
30. Kishore, N., et al., IKK-i and TBK-1 are enzymatically distinct from the homologous enzyme IKK-2: comparative analysis of recombinant human IKK-i, TBK-1, and IKK-2. J Biol Chem, 2002. 277(16): p. 13840-7.
31. Subramanian, A., et al., A Next Generation Connectivity Map: L1000 Platform and the First 1,000,000 Profiles. Cell, 2017. 171(6): p. 1437-1452.e17.
32. Lamb, J., et al., The Connectivity Map: using gene-expression signatures to connect small molecules, genes, and disease. Science, 2006. 313(5795): p. 1929-35.
33. Xu, J. Y., et al., Integrative Proteomic Characterization of Human Lung Adenocarcinoma. Cell, 2020. 182(1): p. 245-261.e17.
34. Fuertes, M. B., et al., Type I interferon response and innate immune sensing of cancer. Trends Immunol, 2013. 34(2): p. 67-73.
35. Corsello, S. M., et al., Discovering the anti-cancer potential of non-oncology drugs by systematic viability profiling. Nat Cancer, 2020. 1(2): p. 235-248.
36. Yang, W., et al., Genomics of Drug Sensitivity in Cancer (GDSC): a resource for therapeutic biomarker discovery in cancer cells. Nucleic Acids Res, 2013. 41(Database issue): p. D955-61.
37. Iorio, F., et al., A Landscape of Pharmacogenomic Interactions in Cancer. Cell, 2016. 166(3): p. 740-754.
38. Garnett, M. J., et al., Systematic identification of genomic markers of drug sensitivity in cancer cells. Nature, 2012. 483(7391): p. 570-575.
39. Hellmann, M. D., et al., Nivolumab plus ipilimumab as first-line treatment for advanced non-small-cell lung cancer (CheckMate 012): results of an open-label, phase 1, multicohort study. Lancet Oncol, 2017. 18(1): p. 31-41.
40. Quint, L. E., et al., CT-guided thoracic core biopsies: value of a negative result. Cancer Imaging, 2006. 6(1): p. 163-7.
41. Casaluce, F., et al., Selumetinib for the treatment of non-small cell lung cancer. Expert Opin Investig Drugs, 2017. 26(8): p. 973-984.
42. Wang, W. W., et al., The efficacy and safety of selumetinib as secondary therapy for late-stage and metastatic non-small cell lung cancer: results from a systematic review and meta-analysis. Ann Transl Med, 2022. 10(10): p. 593.
43. Lebbé, C., et al., Selective Oral MEK1/2 Inhibitor Pimasertib in Metastatic Melanoma: Antitumor Activity in a Phase I, Dose-Escalation Trial. Target Oncol, 2021. 16(1): p. 47-57.
44. Delord, J. P., et al., Selective Oral MEK1/2 Inhibitor Pimasertib: A Phase I Trial in Patients with Advanced Solid Tumors. Target Oncol, 2021. 16(1): p. 37-46.
45. Ripple, M. O., N. Kim, and R. Springett, Acute mitochondrial inhibition by mitogen-activated protein kinase/extracellular signal-regulated kinase kinase (MEK) 1/2 inhibitors regulates proliferation. J Biol Chem, 2013. 288(5): p. 2933-40.
46. Besson, B., et al., Kinome-Wide RNA Interference Screening Identifies Mitogen-Activated Protein Kinases and Phosphatidylinositol Metabolism as Key Factors for Rabies Virus Infection. mSphere, 2019. 4(3).
47. Franklin, J. M. and G. A. Carrasco, Cannabinoid receptor agonists upregulate and enhance serotonin 2A (5-HT(2A)) receptor activity via ERK1/2 signaling. Synapse, 2013. 67(3): p. 145-59.
48. Ciruela, A., et al., Identification of MEK1 as a novel target for the treatment of neuropathic pain. Br J Pharmacol, 2003. 138(5): p. 751-6.
49. Aksamitiene, E., et al., PI3K/Akt-sensitive MEK-independent compensatory circuit of ERK activation in ER-positive PI3K-mutant T47D breast cancer cells. Cell Signal, 2010. 22(9): p. 1369-78.
50. Pelletier, J. P., et al., In vivo selective inhibition of mitogen-activated protein kinase kinase 1/2 in rabbit experimental osteoarthritis is associated with a reduction in the development of structural changes. Arthritis Rheum, 2003. 48(6): p. 1582-93.
51. Favata, M. F., et al., Identification of a novel inhibitor of mitogen-activated protein kinase kinase. J Biol Chem, 1998. 273(29): p. 18623-32.
52. Fukazawa, H., et al., Mitogen-activated protein extracellular signal-regulated kinase kinase (MEK) inhibitors restore anoikis sensitivity in human breast cancer cell lines with a constitutively activated extracellular-regulated kinase (ERK) pathway. Mol Cancer Ther, 2002. 1(5): p. 303-9.
53. Antczak, C., et al., A high-content biosensor-based screen identifies cell-permeable activators and inhibitors of EGFR function: implications in drug discovery. J Biomol Screen, 2012. 17(7): p. 885-99.
54. Wu, W. B., et al., Decreased PGF may contribute to trophoblast dysfunction in fetal growth restriction. Reproduction, 2017. 154(3): p. 319-329.
55. Zhou, X., et al., PQ401, an IGF-1R inhibitor, induces apoptosis and inhibits growth, proliferation and migration of glioma cells. J Chemother, 2016. 28(1): p. 44-9.
56. Qi, B., et al., IGF-1R inhibitor PQ401 inhibits osteosarcoma cell proliferation, migration and colony formation. Int J Clin Exp Pathol, 2019. 12(5): p. 1589-1598.
57. Haura, E. B., et al., A phase II study of PD-0325901, an oral MEK inhibitor, in previously treated patients with advanced non-small cell lung cancer. Clin Cancer Res, 2010. 16(8): p. 2450-7.
58. Rajagopalan, P., et al., Rational combinations of indirubin and arylidene derivatives exhibit synergism in human non-small cell lung carcinoma cells. J Food Biochem, 2019. 43(7): p. e12861.
59. Laurie, S. A., et al., A phase II trial of saracatinib, an inhibitor of src kinases, in previously-treated advanced non-small-cell lung cancer: the princess margaret hospital phase II consortium. Clin Lung Cancer, 2014. 15(1): p. 52-7.
60. Franks, S. E., et al., BMS-754807 is cytotoxic to non-small cell lung cancer cells and enhances the effects of platinum chemotherapeutics in the human lung cancer cell line A549. BMC Res Notes, 2016. 9: p. 134.
61. Yuan, M., et al., SRC and MEK Co-inhibition Synergistically Enhances the Anti-tumor Effect in Both Non-small-cell Lung Cancer (NSCLC) and Erlotinib-Resistant NSCLC. Front Oncol, 2019. 9: p. 586.
62. Ciuleanu, T. E., et al., Randomised Phase 2 study of maintenance linsitinib (OSI-906) in combination with erlotinib compared with placebo plus erlotinib after platinum-based chemotherapy in patients with advanced non-small cell lung cancer. Br J Cancer, 2017. 117(6): p. 757-766.
63. Leighl, N. B., et al., Phase 2 Study of Erlotinib in Combination With Linsitinib (OSI-906) or Placebo in Chemotherapy-Naive Patients With Non-Small-Cell Lung Cancer and Activating Epidermal Growth Factor Receptor Mutations. Clin Lung Cancer, 2017. 18(1): p. 34-42.e2.
64. Liu, X., et al., Morphine promotes the malignant biological behavior of non-small cell lung cancer cells through the MOR/Src/mTOR pathway. Cancer Cell Int, 2021. 21(1): p. 622.
65. Moody, T. W., et al., Endothelin causes transactivation of the EGFR and HER2 in non-small cell lung cancer cells. Peptides, 2017. 90: p. 90-99.
66. Suehiro, Y., et al., Germline copy number variations associated with breast cancer susceptibility in a Japanese population. Tumour Biol, 2013. 34(2): p. 947-52.
67. Hu, M. C., et al., IkappaB kinase promotes tumorigenesis through inhibition of forkhead FOXO3a. Cell, 2004. 117(2): p. 225-37.
68. Yang, J., et al., Conditional ablation of Ikkb inhibits melanoma tumor development in mice. J Clin Invest, 2010. 120(7): p. 2563-74.
69. Pan, F., et al., The novel circ_0028171/miR-218-5p/IKBKB axis promotes osteosarcoma cancer progression. Cancer Cell Int, 2020. 20: p. 484.
70. Bowers, J. L., et al., ATP hydrolysis by ORC catalyzes reiterative Mcm2-7 assembly at a defined origin of replication. Mol Cell, 2004. 16(6): p. 967-78.
71. Forsburg, S. L., Eukaryotic MCM proteins: beyond replication initiation. Microbiol Mol Biol Rev, 2004. 68(1): p. 109-31.
72. Huang, C., et al., Potential Prospective Biomarkers for Non-small Cell Lung Cancer: Mini-Chromosome Maintenance Proteins. Front Genet, 2021. 12: p. 587017.
73. Yuan, J., et al., Multi-Omics Analysis of MCM2 as a Promising Biomarker in Pan-Cancer. Front Cell Dev Biol, 2022. 10: p. 852135.
74. Xiang, J., et al., Levels of human replication factor C4, a clamp loader, correlate with tumor progression and predict the prognosis for colorectal cancer. J Transl Med, 2014. 12: p. 320.
75. Peng, C., et al., Investigation of crucial genes and microRNAs in conventional osteosarcoma using gene expression profiling analysis. Mol Med Rep, 2017. 16(5): p. 7617-7624.
76. Aryal, R. P., et al., Macromolecular Assemblies of the Mammalian Circadian Clock. Mol Cell, 2017. 67(5): p. 770-782.e6.
77. Onyeisi, J. O. S., C. C. Lopez, and M. Götte, Role of Syndecan-4 in breast cancer pathophysiology. Am J Physiol Cell Physiol, 2022.
78. Miller, M. R., et al., Downregulation of dystroglycan glycosyltransferases LARGE2 and ISPD associate with increased mortality in clear cell renal cell carcinoma. Mol Cancer, 2015. 14: p. 141.
79. Day, B. W., et al., The dystroglycan receptor maintains glioma stem cells in the vascular niche. Acta Neuropathol, 2019. 138(6): p. 1033-1052.
80. Liu, L., et al., SLC39A8/Zinc Suppresses the Progression of Clear Cell Renal Cell Carcinoma. Front Oncol, 2021. 11: p. 651921.
81. An, X., et al., An Analysis of the Expression and Association with Immune Cell Infiltration of the cGAS STING Pathway in Pan-Cancer. Mol Ther Nucleic Acids, 2019. 14: p. 80-89.
82. Wang, W., et al., MTA1-TJP1 interaction and its involvement in non-small cell lung cancer metastasis. Transl Oncol, 2022. 25: p. 101500.
83. Moon, S. U., J. H. Kim, and H. G. Woo, Tumor suppressor RBM24 inhibits nuclear translocation of CTNNB1 and TP63 expression in liver cancer cells. Oncol Lett, 2021. 22(3): p. 674.
84. Wu, H., et al., TRAF6 inhibits colorectal cancer metastasis through regulating selective autophagic CTNNB1/β-catenin degradation and is targeted for GSK3B/GSK3β-mediated phosphorylation and degradation. Autophagy, 2019. 15(9): p. 1506-1522.
85. Katoh, M., Multi-layered prevention and treatment of chronic inflammation, organ fibrosis and cancer associated with canonical WNT/β-catenin signaling activation (Review). Int J Mol Med, 2018. 42(2): p. 713-725.
86. Rivas, M. A., et al., Smc3 dosage regulates B cell transit through germinal centers and restricts their malignant transformation. Nat Immunol, 2021. 22(2): p. 240-253.
87. Yano, K., et al., Regulation of breast cancer resistance protein and P-glycoprotein by ezrin, radixin and moesin in lung, intestinal and renal cancer cell lines. J Pharm Pharmacol, 2020. 72(4): p. 575-582.
88. Vasaikar, S. V., et al., LinkedOmics: analyzing multi-omics data within and across 32 cancer types. Nucleic Acids Res, 2018. 46(D1): p. D956-D963.
89. Aran, D., Z. Hu, and A. J. Butte, xCell: digitally portraying the tissue cellular heterogeneity landscape. Genome Biol, 2017. 18(1): p. 220.
90. Hall, M., et al., The WEKA data mining software. ACM SIGKDD Explorations Newsletter, 2009. 11(1): p. 10-18.
91. Addison, J. B., et al., Functional hierarchy and cooperation of EMT master transcription factors in breast cancer metastasis. Mol Cancer Res, 2021.
92. Guo, L., B. Cukic, and H. Singh, Predicting Fault Prone Modules by the Dempster-Shafer Belief Networks. Proceedings of 18th IEEE International Conference on Automated Software Engineering (ASE'03), 2003: p. 249-252.
93. Guo, N. L., et al., A novel network model identified a 13-gene lung cancer prognostic signature. Int J Comput Biol Drug Des, 2011. 4(1): p. 19-39.
94. Guo, N. L. and Y. W. Wan, Pathway-based identification of a smoking associated 6-gene signature predictive of lung cancer risk and survival. Artif Intell Med, 2012. 55(2): p. 97-105.
95. Ye, Q., et al., Immune-Omics Networks of CD27, PD1, and PDL1 in Non-Small Cell Lung Cancer. Cancers (Basel), 2021. 13(17).
96. Walter, R. F., et al., ACTB, CDKN1B, GAPDH, GRB2, RHOA and SDCBP Were Identified as Reference Genes in Neuroendocrine Lung Cancer via the nCounter Technology. PLOS One, 2016. 11(11): p. e0165181.
97. Saviozzi, S., et al., Selection of suitable reference genes for accurate normalization of gene expression profile studies in non-small cell lung cancer. BMC Cancer, 2006. 6: p. 200.
98. Chang, Y. C., et al., Differential expression patterns of housekeeping genes increase diagnostic and prognostic value in lung cancer. PeerJ, 2018. 6: p. e4719.
99. Shannon, P., et al., Cytoscape: a software environment for integrated models of biomolecular interaction networks. Genome Res, 2003. 13(11): p. 2498-504.
100. Meyers, R. M., et al., Computational correction of copy number effect improves specificity of CRISPR-Cas9 essentiality screens in cancer cells. Nat Genet, 2017. 49(12): p. 1779-1784.
101. Dempster, J. M. R., J.; Kazachkova, M.; Pan, J.; Kugener, G.; Root, D. E.; Tsherniak, A., Extracting Biological Insights from the Project Achilles Genome-Scale CRISPR Screens in Cancer Cell Lines. bioRxiv, 2019.
102. DepMap, DepMap 20Q2 Public 2020: In Broad: figshare.
103. McFarland, J. M., et al., Improved estimation of cancer dependencies from large-scale RNAi screens using model-based normalization and data integration. Nat Commun, 2018. 9(1): p. 4610.
104. Chen, J., et al., ToppGene Suite for gene list enrichment analysis and candidate gene prioritization. Nucleic Acids Res, 2009. 37(Web Server issue): p. W305-11.
105. Nusinow, D. P., et al., Quantitative Proteomics of the Cancer Cell Line Encyclopedia. Cell, 2020. 180(2): p. 387-402.e16.
106. Team, R., RStudio: Integrated Development Environment for R. 2020, RStudio, PBC.: Boston, MA.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method of treating a patient having non-small cell lung cancer comprising administering to said patient a therapeutically effective amount of a compound selected from the group consisting of PD-198306 or pharmaceutically acceptable salts thereof, U-0126 or pharmaceutically acceptable salts thereof, ZM-306416 or pharmaceutically acceptable salts thereof, and PQ-401 or pharmaceutically acceptable salts thereof, for treating non-small cell lung cancer in said patient.

2. The method of claim 1 including wherein said compound is PD-198306 or pharmaceutically acceptable salts thereof.

3. The method of claim 1 including wherein said compound is U-0126 or pharmaceutically acceptable salts thereof.

4. The method of claim 1 including wherein said compound is ZM-306416 or pharmaceutically acceptable salts thereof.

5. The method of claim 1 including wherein said compound is PQ-401 or pharmaceutically acceptable salts thereof.

6. A method of treating a patient having non-small cell lung cancer comprising administering to said patient a therapeutically effective amount of a composition comprising a compound selected from the group consisting of PD-198306 or pharmaceutically acceptable salts thereof, U-0126 or pharmaceutically acceptable salts thereof, ZM-306416 or pharmaceutically acceptable salts thereof, and PQ-401 or pharmaceutically acceptable salts thereof, and a therapeutically acceptable pharmaceutical carrier, for treating non-small cell lung cancer in said patient.

7. A method for producing an immune response in a patient having non-small cell lung cancer comprising administering to said patient a therapeutically effective amount of a compound selected from the group consisting of PD-198306, U-0126, ZM-306416, and PQ-401, for producing an immune response in said patient.

8. A method for producing an immune response in a patient having non-small cell lung cancer comprising administering to said patient a therapeutically effective amount of a composition comprising a compound selected from the group consisting of PD-198306, U-0126, ZM-306416, and PQ-401, and a therapeutically acceptable pharmaceutical carrier, for producing an immune response in said patient.

9. A method of treating a patient having non-small cell lung cancer comprising administering to said patient a therapeutically effective amount of a compound that is a MEK1/2 inhibitor selected from the group consisting of PD-198306 and U-0126.

10. A method of treating a patient having non-small cell lung cancer comprising administering to said patient a therapeutically effective amount of a composition comprising a compound that is a MEK1/2 inhibitor selected from the group consisting of PD-198306 and U-0126, and a therapeutically acceptable pharmaceutical carrier.

11. A method of treating a patient having non-small cell lung cancer comprising administering to said patient a therapeutically effective amount of a compound that is a VEGFR inhibitor that is ZM-306416.

12. A method of treating a patient having non-small cell lung cancer comprising administering to said patient a therapeutically effective amount of a composition comprising a compound that is a VEGFR inhibitor that is ZM-306416, and a therapeutically acceptable pharmaceutical carrier for treating a patient having non-small cell lung cancer.

13. A method of treating a patient having non-small cell lung cancer comprising administering to said patient a therapeutically effective amount of a compound that is a IGF-1R inhibitor that is PQ-401.

14. A method of treating a patient having non-small cell lung cancer comprising administering to said patient a therapeutically effective amount of a composition comprising a compound that is a IGF-1R inhibitor that is PQ-401 and a therapeutically acceptable pharmaceutical carrier for treating a patient having non-small cell lung cancer.

15. A method of diagnosis and screening of non-small cell lung cancer comprising using protein expression levels of one elected from the group consisting of ABCC4, DAG1, and SLC39A8 in patient samples, of ABCC4 and DAG1 in patient samples, of ABCC4 and SLC39A8 in patient samples, of DAG1 and SLC39A8 in patient samples, of ABCC4 in patient samples, of DAG1 in patient samples, and of SLC39A8 in patient samples.

16. A method of diagnosis and screening of non-small cell lung cancer comprising using mRNA expression levels of ABCC4, CCL19, CD27, DAG1, FUT7, and ZNF71 in patient samples.
